(19) United States
(12) Patent Application Publication
(10) Pub. No.: US 2004/0210036 A1
(43) Pub. Date: Oct. 21, 2004

(54) Peptide substrate libraries

(75) Inventors: Brian Dwyer, San Diego, CA (US); John Heimbold, San Diego, CA (US); Siobhan Miick, San Diego, CA (US)

Correspondence Address: MORGAN LEWIS & BOCKIUS LLP, 1111 PENNSYLVANIA AVENUE NW, WASHINGTON, DC, 20004, US

(73) Assignee: NANOGEN, INC.

(21) Appl. No.: 10/413,572

(22) Filed: Apr. 15, 2003

(51) Int. Cl. G01N 33/53; C07K 14/00; G01N 33/543

(52) U.S. Cl. 530/350; 435/7.1; 436/518

(57) ABSTRACT

Peptide libraries containing peptides having the same net charge or same like charge are described, which may be used to screen for substrates of kinases and phosphatases and any other enzyme that causes a difference in net charge in a suitable substrate. The libraries are designed using representative amino acids for one or more classes of amino acids, thereby reducing the number of members in the peptide library. Reducing the number of peptides in the library to a manageable size permits the peptides to be segregated into individual wells of a microtiter plate, where the sequences of suitable substrates are immediately ascertainable upon exposure to enzyme by detecting the position of charge inverted peptide substrates in the plate.

### FIELD OF INVENTION

The invention relates to the design and synthesis of peptide substrate libraries where all the peptides in the library have the same charge, the same like charge, or a neutral charge. Such libraries are particularly useful for identifying substrates for protein kinases and phosphatases, or for identifying any substrate that accepts or donates an ion or charged group when acted upon by an enzyme, thereby resulting in a gain or loss of net charge. In some embodiments, an algorithm that applies a defined set of constraints optionally contained on a computer readable medium may be used to generate the peptide library. These constraints may include, in addition to setting a charge value, defining the numbers and types of amino acids permitted in each variable position of each peptide. By minimizing the number of possible amino acids in the variable positions of the peptides, the number of unique peptides in a particular library may be reduced to a level that can be readily synthesized and screened for enzyme activity.

Sequence Listing Submission on Compact Disc

The Sequence Listing submitted concurrently herewith on compact disc under 37 C.F.R. §§1.821(c) and 1.821(e) is herein incorporated by reference in its entirety. Three copies of the Sequence Listing, one on each of three compact discs are provided. Copy 1 and Copy 2 are identical. Copies 1 and 2 are also identical to the CRF. Each electronic copy of the Sequence Listing was created on Jul. 14, 2003 with a file size of 1640 KB. The file names are as follows: Copy 1—Nanogen 5002US.txt; Copy 2—Nanogen 5002US.txt; CRF—Nanogen 5002US.txt.

### BACKGROUND OF INVENTION

Protein kinases are enzymes that regulate a great variety of physiological and pathophysiological processes, including signal transduction (Yang et al., 2003, Mutat. Res. 543(1): 31-58), transcriptional regulation (Dunn et al., 2002, Cell Signal. 14(7): 585-93), cell motility (Knaus and Bokoch, 1998, Int. J. Biochem. Cell Biol. 30(8): 857-62) cell division (Johnson and Lapadat, 2002, Science 298(5600): 1911-2), differentiation (Rebay, 2002, Dev. Biol. 251(1): 1-17), apoptosis (Shohat et al., 2002, Biochim Biophys Acta 1600(1-2): 45-50), ion channels (Wang et al., 2002, Trends Cardiovasc. Med. 12(3): 138-42) and cell-cell communications (Schmidt et al., 1993, Semin. Cell Biol. 4(3): 161-73). The near-completion of the human genome sequence has allowed the identification of over 500 human protein kinases constituting about 1.7% of all human genes (Manning et al., 2002, Science 298:1912). Yet, despite the large number of kinases identified, few of the kinases have been characterized with respect to substrate specificity.

Protein kinases catalyze the phosphorylation of their substrate proteins by transferring a phosphoryl group onto an amino acid residue in the substrate protein using adenosine triphosphate (ATP) as the phosphate donor. Substrate specificity is determined to a large extent by the amino acids on either side of the phosphorylation site. In animal cells, serine, threonine and tyrosine are the most commonly phosphorylated amino acid residues. Phosphatases, in turn, are enzymes that remove phosphoryl groups from substrate proteins. Deregulation of either protein kinase or protein phosphatase activity can contribute to the pathogenesis of many diseases, including cancer, diabetes, rheumatoid arthritis and hypertension (Zhang, 2002, Annu. Rev. Pharmacol. Toxicol. 42: 209-34).

Traditionally, substrates of protein kinases are discovered by sequencing phosphorylated cellular proteins. After a substrate is identified, assays for protein kinases may be performed using synthetic peptide substrates that contain the particular sequence phosphorylated by the protein kinase of interest. The identification and sequencing of proteins require a large amount of work before a substrate for a certain protein kinase can be identified.

The introduction of peptide libraries lessened the amount of work required for finding kinase substrates. Libraries provide a convenient way for identifying suitable substrates and also provide a rich source of potential compounds for pharmaceutical leads. Peptide libraries may encompass millions of different peptides, thereby supplying a repertoire of structural diversity and allowing many potential substrates to be screened at one time. The large diversity and simultaneous screening capacity facilitated by the use of peptide libraries increases the chances of finding candidate substrate sequences.

Many methods have been devised for preparing and screening substrate libraries. For instance, one method is to use multiple kinase substrate libraries with either one or two degenerate, or variable, positions. Synthetic peptides are constructed and individual amino acids are replaced one by one in order to determine the importance of the particular amino acid on the phosphorylation site. Till and colleagues describe this approach with two libraries each containing only one degenerate position in a peptide sequence of 7 or 13 amino acid residues (Till et al., 1993, J. Biol. Chem. 269: 7423-28). Pinnila et al. also describes constructing six synthetic peptide libraries composed of 18 peptide mixtures with a single degenerate position (1992, Biotechniques 13: 901-06). Such dual or multiple library approaches, however, are expensive and time-consuming. Each amino acid is individually altered within a phosphorylation site to determine its importance, which permits only limited peptides to be screened. Therefore, finding an optimal substrate sequence is unlikely.

Other methods have been developed that allow the screening of random synthetic peptide libraries containing millions of different peptides having numerous degenerate positions. For example, Wu and colleagues describe screening random synthetic combinatorial peptide libraries on beads where each bead carries only one peptide entity (Wu et al., 1994, Biochem. 33: 14825-14833). The method uses a total of nineteen natural amino acids and each library contains peptides of five to seven amino acids and 500,000 beads. Thus, the number of permutations ranges from 19⁵ to 19⁷. However, since the sequence of the peptide on any particular bead is not known, beads containing phosphorylated peptides must be separated and sequenced to identify phosphorylated peptides.

Similarly, Songyang, Cantley and colleagues describe a technique using a soluble peptide library of 15mer peptides containing eight degenerate positions adjacent to serine or tyrosine to evaluate substrate motifs of several serine/threonine and tyrosine kinases (Songyang et al., 1994, Current Biol. 4: 973-82; see also U.S. Pat. No. 5,532,167). However, again, since all peptides in the library are mixed together in solution, phosphorylated peptides must first be separated using either a ferric or mercury column in the case of thio-phophorylated peptides, or by using antibody affinity column. The separated peptides are then sequenced as a bulk population to determine a putative substrate “motif.”

Tegge et al. describe the use of a library of octamers on cellulose paper, dubbed the “SPOT-Method,” using mixtures of all 20 amino acids, divided into 400 sublibraries containing 6.4×10⁷ sequences (Tegge et al., 1995, Biochem. 34: 10569-77; Tegge and Frank, 1998, Methods in Mol. Biol. Vol. 87: Combinatorial Peptide Library Protocols). While sequencing of phosphorylated peptides is not required in the SPOT-Method, the method is an iterative technique that only permits the evaluation of two positions in the peptide sequence with each use, and therefore must be repeated until all amino acids in the peptide may be evaluated. In other words, the best amino acid combination identified using a particular array is used in all the peptides in the following array, which means the libraries used in this technique are extremely time-consuming to produce since the synthesis of one library depends on the results from the one before.

In an effort to reduce library size, some groups have proposed using “doping” strategies rather than complete randomization, where site-specific variations are biased to include only a certain subset of amino acids per position (Tomandl et al., 1996, J. Computer-Aided Molecular Design, 1997, 11:29-38; Ostergaard and Holmes, 1997, J. Peptide Sci. 3:123-32). However, this technique requires some beginning knowledge regarding the sequence of the target peptide substrate. Other groups have proposed the use of virtual screening methods to predict which molecules in a virtual library are most likely to be active before a combinatorial library is synthesized (Sheridan et al., J. Mol. Graphics and Modelling, 2000, 18: 320-334). This technique requires some knowledge regarding the structure or sequence of the enzyme of interest. Thus, there remains a significant need for methods to reduce the complexity and size of peptide libraries, particularly for the synthesis of libraries for screening for kinase and phosphatase substrate specificity.

What is needed in the art is a means of producing a peptide library for screening kinase substrates that is convenient to use, i.e., does not require sequencing or other manipulation to obtain a result, and one that does not involve the cost of synthesizing millions of individual peptide sequences or the time required for synthesizing successive more focused libraries as more information about a substrate is obtained. Further, what would be useful is a means of defining and presenting a library such that the sequence of all members is known beforehand and the sequence of a suitable substrate is immediately determinable, where the library is presented with a manageable number of representative members.

### SUMMARY OF THE INVENTION

The present invention provides methods of designing peptide libraries of manageable size, wherein each of the individual peptides of the libraries has the same net charge, the same like net charge, or a neutral charge. For instance, where each of the peptides in the library has a net charge of +1, peptides phosphorylated in the presence of a kinase may be identified by virtue of a charge inversion from +1 to −1, since addition of a phosphoryl group to a peptide adds a net −2 charge. In contrast, where each of the peptides in the library has a net charge of −1, peptides that have been dephosphorylated by a phosphatase may be identified by virtue of a charge inversion from −1 to +1. The peptide libraries of the invention may be designed by ensuring that the number of positively charged amino acids in each peptide taken together with the number of negatively charged amino acids in each peptides equals the desired net charge for peptides in the given library. An algorithm for designing peptide sequences employing this constraint, in addition to possible other constraints, is also provided. Algorithms may be provided on a computer readable medium storing computer executable instructions for designing the libraries of the invention.

The peptide libraries of the present invention may be confined to a manageable size by using a defined subset of amino acids as representatives of the different classes of amino acids. For instance, by reducing the number of possible amino acids in each variable position to 5 representatives of the different types of amino acids, i.e., positively and negatively charged, neutral, hydrophobic, and bending, the number of members in the library is drastically reduced as compared to a library where each of the natural twenty amino acids is included at each variable position. Further variability may be introduced by using binary subsets of representative amino acids when synthesizing the peptide library. Restricting the size of the libraries to a manageable size permits high throughput screening using microtiter plates that have been specially designed for electrophoretic manipulation.

In addition to methods for designing peptide libraries, the present invention provides peptide libraries comprising a plurality of different peptide molecules, wherein each peptide molecule in the library has the same net charge or a neutral charge. While the peptides of the libraries of the invention may be contained in a collection in solution, the libraries are particularly useful when individual peptide molecules, or mixtures of peptide molecules having at least one variable position, are segregated. In some embodiments, among others, individual peptide molecules or variable mixtures thereof are segregated into individual wells of one or more microtiter plates, which may then be fit into an electrophoretic apparatus for detection of charge inverted peptides. In such embodiments, because the peptides or mixtures having peptides of similar sequence have already been segregated into known positions in the microtiter dish, the sequence of active substrates is immediately known after analysis of the electrophoretic microtiter plate. No further sequencing is required to determine the sequence of a peptide substrate or a peptide motif. Peptide libraries may be supplied to the user in microtiter plates, or in kits comprising the microtiter plates in addition to various other reagents.

The peptide libraries of the present invention may be used in methods for identifying an ion-donating or ion-accepting peptide substrate of an enzyme, or a phosphoryl-donating or phosphoryl-accepting peptide substrate of a kinase or phosphatase, comprising (a) contacting the peptide library of the invention with the enzyme under conditions that allow for ion transfer to or from peptides that are substrates for said enzyme; and (b) identifying peptides in the library that have been modified by the enzyme. In some embodiments, among others, the peptides in the library are attached to a detectable label, such as a fluorophore. In microtiter plate embodiments, peptides having undergone a charge inversion may be captured onto a matrix upon application of an electric field, and detected by virtue of the attached label. Alternatively, fluorescent peptides may be visualized following electrophoresis in a planar agarose gel.

### BRIEF DESCRIPTION OF THE DRAWINGS

FIG. 1. Graph depicting fluorescence signal intensity versus peptide library well for PKAalpha library screen.

### DETAILED DESCRIPTION OF THE INVENTION

As described above, the present invention provides for the synthesis of peptide libraries of a manageable size that may be screened, for instance, using a format where individual peptides are segregated into individual wells of a microtiter plate. To illustrate how quickly a library of short peptides sequences can grow to a very large, unmanageable number, a peptide library having a given number of variable positions has mⁿ different possible members where m is the number of different amino acids that could be in each variable position and n is the number of variable positions. Thus, the total number of members of a peptide library containing peptides with six variable positions that may be filled with any of 20 amino acids is 20⁶ or 64,000,000. This is quite a large number of peptides that would be difficult to synthesize let alone screen individually. If, however, the number of possible amino acids in the variable positions is reduced to 5 representatives of the different types of amino acids, i.e., positively and negatively charged, neutral, hydrophobic, and bending, then the number of members in the library is reduced to 5⁶ or 15,625.

The libraries of the present invention are suitable for screening for kinase or phosphatase substrates, or for any enzyme substrate where the enzyme creates a charge inversion from positive to negative or vice versa. For instance, a library containing peptides having a +1 or neutral charge may be used to identify suitable kinase substrates by identifying those that exhibit a negative charge following exposure to the kinase. In contrast, a peptide library containing members having a −1 or neutral charge permits identification of phosphatase substrates as those peptides that exhibit a positive charge following exposure to the phosphatase.

An algorithm may be used in the methods of the present invention to design peptides of the same charge using representative amino acids to limit library size as described above. For example, where peptides having a +1 charge are desired, i.e., for screening kinase substrates, the algorithm may be constrained to identify peptides where the number of basic residues (arginine or lysine) minus the number of acidic residues (aspartic acid or glutamic acid) must equal +1. Since no other residues or moieties in the peptides are charged, this constraint ensures that all peptides in the library have a charge of +1. In one embodiment, among others, the amino acids for the variable positions are limited to just five of the twenty naturally occurring amino acids, i.e., arginine (R) as the positively charged representative, glutamic acid (E) as the negatively charged representative, alanine (A) as the neutral representative, leucine (L) as the hydrophobic representative, and proline (P) to introduce a bend in the backbone of the peptide.

As is known, cysteine and tyrosine could become charged above pH 8 and 10, respectively. Therefore, these amino acids may be used if reaction conditions permit. Alternatively, cysteine may be omitted from the peptide library, and tyrosine may be used only when serving as a phosphoryl-group acceptor or donor, taking into account the charge in relation to the pH when setting the constraints for the library design.

The phosphoryl-group acceptor residue for a kinase substrate library (or phosphoryl-group donor residue for a phosphatase library) could also be degenerately designed to include serine, threonine, tyrosine, histidine, aspartate or lysine so that the library could be used to screen any protein kinase (or phosphatase). Alternatively, the phosphate acceptor (or donor) residue may be defined narrowly for the screening of a specific kinase or family of kinases (or phosphatases). Where, aspartate or lysine is used as the phosphoryl-group donor or acceptor, the peptide library may be designed accounting for the positive and negative charges of these amino acids, respectively. When designing these libraries, the algorithm constraints for the other amino acid members of the library may be modified in order to achieve a library of peptides with the desired charge.

The position of the phosphate acceptor or donor residue need not be limited to the central position. For instance, the phosphate acceptor or donor residue may be shifted towards either terminus to probe more stringently the specificity of the enzyme in the amino terminal or carboxy terminal portion of the substrate. Indeed, two libraries may be employed where each is shifted towards the amino or carboxy terminus, respectively, to further define the specificity of the enzyme.

In addition, the library may be designed to have multiple phosphohorylation sites, i.e., for screening one kinase that phosphorylates multiple sites. For example, PKCbeta has 4 phosphorylation sites, that are phosphorylated sequentially. In order to phosphorylate the second site, the first site must be phosphorylated and may be part of the recognition sequence. In such instances, phosphoserine, phosphothreonine and/or phosphotyrosine may be included in the types of amino acids in the library, and the constraints of the algorithm may be adjusted accordingly to select for multiply phosphorylated or dephosphorylated peptides following charge inversion.

As discussed below in greater detail, the algorithm used to design the peptide libraries of the invention may contain additional constraints to further reduce the number of peptides in the library. For instance, the number of peptides that are possible for a +1 library after applying the following constraints—(# R)−(# E)=+1, # R<3, # L<3, and # P<2—reduces the library size from 15,625 (or 5⁶ members as discussed above) to 1,566. See Table 5 below for an exemplary library of peptides generated using such an algorithm, using the amino acids arginine (R), glutamic acid (E), alanine (A), leucine (L), and proline (P), with a serine residue fixed in the center position. This collection would fit in a set of five 384-well plates, for instance, and could readily be screened in a day. Since each peptide is contained separately from the others and each well may be pre-fitted with a particular peptide sequence, the sequences of suitable substrates are immediately ascertainable. Once identified as a suitable substrate the peptide may be used directly in enzyme assays. Alternatively, suitable peptides can be used as a starting point for the synthesis of analogs that contain other amino acids in order to optimize the activity of the peptide substrate, or as a starting point to design enzyme inhibitors.

In order to increase the diversity of the peptide library without increasing the number of individually pure peptides, directed mixtures can be prepared where degeneracy is limited to one or two positions. Alternatively, instead of selecting one of several amino acids from a particular class of proteogenic amino acids as a representative of the class, for instance, a mixture of amino acids may be used (e.g., leucine, isoleucine, phenylalanine and/or valine may be used as representatives of the hydrophobic class). In some embodiments described herein, binary mixtures of representative classes are used. Identification of the phosphorylated product within such a mixture may be accomplished using mass spectrometry. Alternatively, an unnatural amino acid such as homoalanine (2-aminobutyric acid) could be used that contains attributes of more than one class. For instance, instead of using the five amino acids alanine, leucine, arginine, glutamic acid and proline for a library of 6mer peptides, the unnatural amino acid homoalanine may be substituted for alanine and leucine, thereby reducing the number of members of the library from 5⁶ (or 15,650) to 4⁶ (or 4,096).

Further details relating to peptide library construction and methods of using the libraries for substrate screening are described in more detail in the sections to follow.

Definitions

The present invention provides peptide libraries comprising a plurality of different peptide molecules, wherein each peptide molecule in the library has the same net charge, same like charge or a neutral charge. “Same like charge” means that all peptides are either positively charged or negatively charged, but the peptides do not necessarily have to have the same net (total) charge. A “neutral” charge is a net charge of zero, i.e., meaning that the peptide is neither positively nor negatively charged.

A “plurality” with regard to the size of the inventive peptide libraries means more than about 5 or 6 (or 8 for binary applications), or more than about 10, 20, 30, 40, 50, 75, 100, 150, 200, 250, 300, 350, 400, 450, 500, etc. In some embodiments, the libraries of the invention may comprise, for instance, at least about 1000, 1500, 2000, 3000, 5000, 10,000, 15,000 or more peptide molecules.

The peptides in the libraries of the present invention comprise peptides having at least one variable position in relation to one another. “Variable” means degenerate at a particular position, in contrast to “distinct” sequences that are all different and may or may not be degenerate at a given position.

As used herein, “charged” means having a positive or negative total charge. “Charge inversion” of a peptide substrate means that the charge of the peptide is changed to the opposite charge if the peptide is acted upon during the course of the assay. For instance, charge inversion of a positively charged peptide would yield a negatively charged peptide, and charge inversion of a negatively charged peptide would yield a positively charged peptide. It should be understood that charge may vary as a function of pH, and that the charge of the substrate should be designed so as to facilitate separation techniques and the detection of charge inversion when acted upon by the enzyme under pH conditions suitable for the particular enzyme to be tested. The skilled artisan may also design similar assays based on an increase or decrease in charge rather than a charge inversion, using the concepts disclosed herein.

Being of the same charge or same like charge, the peptide libraries of the present invention are particularly suited for screening for substrates of enzymes that either transfer or remove ions or charged groups to or from peptide substrates. An “ion” is defined as an atom or group of atoms that has acquired a net electric charge by gaining or losing one or more electrons. Thus, an “ion-accepting” amino acid is an amino acid in a peptide substrate that accepts a charged atom or group of atoms upon exposure of an enzyme. An “ion-donating” amino acid is an amino acid in a peptide substrate that donates a charged atom or group of atoms upon exposure to an enzyme.

The peptides of the invention are particularly useful for screening for substrates of protein kinases and phosphatases. A “kinase” is any enzyme that transfers a phosphoryl group from ATP to a peptide substrate, thereby resulting in a change in net charge of −2. A phosphatase is any enzyme that removes a phosphoryl group from a peptide substrate, thereby resulting in a change in net charge of +2. While in some instances in the present invention, the transfer of a phosphoryl group has been likened to the transfer of an ion to the extent that a change in net charge occurs in either case, the transfer of a phosphoryl group via the action of a kinase forms one covalent bond (CH₂—O—P) as well as several hydrogen bonds and exhibits a difference in stability as compared to other types of ion exchange. Thus, a “phosphoryl accepting” amino acid is defined as an amino acid that becomes phosphorylated through the action of a kinase, and a “phosphoryl donating” amino acid is defined as an amino acid that donates a phosphoryl group upon exposure to a phosphatase. Where the libraries of the present invention are to be used for screening for phosphatase activity, or for enzyme substrates that contain ion-donating amino acids, the substrates should be synthesized using amino acids carrying the phosphoryl or ion groups of interest.

Further terms of the invention are defined in the text to follow.

Peptide Library Design

As described above, the invention provides for a peptide library comprising individual peptide sequences having the same charge or same like charge such that peptides acted upon by an enzyme of interest may be identified by virtue of a change in net charge. Any of the naturally occurring twenty amino acids may be used to design and synthesize the peptides of the invention. Table 1 shows the twenty natural amino acids and their three-letter abbreviations and canonical one-letter abbreviations.

TABLE 1
Twenty natural amino acids

| | THREE-LETTER | ONE-LETTER |
|---|---|---|
| AMINO ACIDS | ABBREVIATIONS | ABBREVIATIONS |
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As also described above, in order to limit the number of peptides in a library to a manageable size, i.e., where each peptide may be segregated into individual wells of a microtiter plate and separately screened for substrate activity, one or more amino acids may be chosen as a representative of a particular class of amino acids. Thus, by using that representative amino acid or amino acid mixture in all instances to represent that particular class of amino acids, the number of peptides in the library is reduced. As indicated in Table 2, there are preferably five main amino acid classes for creating libraries for screening for kinase and phosphatase activities: basic (B), acidic (A), hydrophobic (U), spacing (J), phosphoryl-accepting or -donating (O), and bending (P). The hydrophilic amino acids (N,Q) have been included in the spacing class, however, this group could form a separate class depending on the preference of the user. Table 2 indicates the amino acid classes and their noncanonical single-letter abbreviations, and the individual proteogenic amino acids in each class. The individual proteogenic amino acids are abbreviated using the canonical one-letter code shown in Table 1. For example, the amino acid lysine (K) is a basic amino acid, and thus, is placed in the basic amino acid class (B). In some embodiments, however, lysine may also be a phosphoryl-donating or -accepting residue, and may also be in class O. Exemplary representative amino acids from each class are also shown, as are representative binary mixtures. However, any of the amino acids from a designated may be used as a representative in the peptide libraries of the invention.

TABLE 2
Amino acid classes

| | SINGLE-LETTER | PROTEOGENIC | REPRESENTATIVE | BINARY |
|---|---|---|---|---|
| AMINO ACID CLASS | ABBREVIATIONS | MEMBERS | MEMBER | MIXTURE |
| Basic | B | K, R, H | R | K, R |
| Acidic | Z | D, E | E | D, E |
| Hydrophobic | U | V, L, M, I, F, Y, W | L | V, L |
| Spacing | J | N, Q, G, A, | A | G, A |
| | | homoalanine | | |
| Phosphate-Accepting | O | S, T, Y, H, D, K | S | S, T |
| or-Donating | | | | |
| Bending | P | P | P | P only |

Some libraries of the present invention are formed using a binary mixture of amino acids as a representative of an amino acid class. For instance, rather than using just glutamic acid (E) as a representative of the acidic class, the binary mixture of aspartic acid (D) and glutamic acid (E) may be used. In embodiments where binary mixtures of amino acids are used, or any mixtures containing more than one amino acid, the single letter code for an amino acid class may be used to describe particular positions in the peptide sequence. To illustrate, an exemplary peptide , when synthesized using a mixture of acidic amino acids rather than just glutamic acid (E), would be designated RRZSPLLG (SEQ ID No. 1567) instead of RRESPLLG (SEQ ID No. 1). Similarly, when this peptide is synthesized with a mixture of phosphate-accepting or donating amino acids instead of just serine (S), the peptide sequence would be designated RREOPLLG (SEQ ID No. 1568) instead of RRESPLLG (SEQ ID No. 1).

Using a mixture of amino acids to represent a particular class according to the present invention means that any of the representative amino acids may be included at the position designated for the class. Alternatively, all of the amino acids of the representative mixture may be included at the designated position, resulting in a sub-population of related peptide sequences in a single well of a microtiter plate. For example, where a binary mixture of acidic amino acids is used, i.e., D and E, the well of the microtiter plate corresponding to the peptide sequence RRZSPLLG (SEQ ID No. 1567) may be designed to contain either one of the peptides RRESPLLG (SEQ ID No. 1) or RRDSPLLG (SEQ ID No. 1569). Alternatively, the well may contain both of the peptides RRESPLLG (SEQ ID No. 1) and RRDSPLLG (SEQ ID No. 1569). Where wells contain a mixture of related peptides, or a peptide containing an unknown representative of a particular class, mass spectrometry, or other means, including direct sequencing, may be used to determine which species underwent charge inversion upon exposure to the enzyme of interest.

Representative amino acids or mixtures thereof may be strategically chosen based on key properties of amino acids. For instance, as shown in Table 3, of the basic amino acid side chains, histidine has the side chain with the lowest pKa value and is therefore neutral at around physiological pH. Both lysine (K) and arginine (R), however, are highly polar and hydrophilic amino acids that are positively charged, and thus would be preferable for synthesizing the peptides of the present invention.

TABLE 3
Class B: Basic amino acids

| | SINGLE-LETTER | TYPICAL |
|---|---|---|
| AMINO ACIDS | ABBREVIATIONS | pKa VALUES |
| Lysine | K | 10.0-10.8 |
| Arginine | R | 12.0-12.5 |
| Histidine | H | 6.1-6.5 |

Table 4 shows amino acid class Z, the acidic amino acids with negatively charged polarity, and their side chain pKa values. Aspartic acid and glutamatic acid are both negatively charged at physiological pH. These strongly polar residues interact favorably with solvent molecules, and both are preferred as acidic class representatives.

TABLE 4
Class Z: Acidic amino acids

| | SINGLE-LETTER | TYPICAL |
|---|---|---|
| AMINO ACIDS | ABBREVIATIONS | pKa VALUES |
| Aspartic acid | D | 3.9-4.4 |
| Glutamic acid | E | 4.1-4.6 |

Hydrophobic amino acids (class U) have a tendency to be found inside a protein and form clusters, and thus, water-soluble proteins are stabilized by the coming together of the hydrophobic side chains to avoid contact with water. The highly hydrophobic amino acids include isoleucine (I), leucine (L), methionine (M), phenylalanine (F), tryptophan (W), tyrosine (Y), and valine (V), as shown in Table 2. Methionine and tryptophan might also be omitted because they are rarely recognized as substrates. Tyrosine is not preferable as a class representative because it is a phosphate-accepting amino acid (particularly if one is screening for substrates of tyrosine phosphatases). Phenylalanine, an aromatic amino acid, is less preferable as it is non-polar and highly hydrophobic, however, certain kinases may have stringent requirements for Phe as a recognition element. Of the remaining hydrophobic amino acids in the class, leucine (L) and valine (V), two hydrophobic aliphatic amino acids, are most preferred as acidic class representatives.

The spacing amino acid class (J) is used as a non-polar, uncharged amino acids for spacing in the backbone of the peptide sequences. Alanine (A), a neutral amino acid, is also preferred as a spacing amino acid because it has the smallest side chain, with the exception of glycine.

Non-natural amino acids can also be incorporated into synthetic libraries of the present invention. For instance, the non-natural amino acid, homoalanine (2-aminobutyric acid), may be used as a spacing amino acid because it contains a subset of substituents present in all of the amino acids except glycine and alanine. Homoalanine is a short, non polar unnatural amino acid. The addition of homoalanine further reduces the number of possible sequences, as homoalanine may be a replacement for both alanine and leucine. Noravaline may be a suitable replacement for a natural hydrophobic amino acid. Amino isobutyric acid (Aib) or cycloleucine may be used for the bending class. Ornithine and homoarginine may be used as representatives of the basic group.

When used to screen for kinase or phosphatase substrate activity, the peptides of the libraries of the invention are further designed to contain a phosphoryl group acceptor or donor (S/T/Y/HID/K) (class O). The phosphoryl accepting amino acids, serine (S), threonine (T), tyrosine (T), histidine (H), aspartate (D) and lysine (K) may be used either as individual residues, or a combination thereof. Serine (S) and threonine (T) contain aliphatic hydroxyl groups that make these amino acids more hydrophilic and more reactive than hydrophobic amino acids. Tyrosine, however, is generally hydrophobic. Depending on the type of kinase, different amino acid sequences with different combinations of phosphoryl-accepting amino acids may be used. For example, the kinase may be a protein serine/threonine specific kinase, therefore, serine or threonine is used in the peptide library. Or tyrosine would be used in peptide sequences, if a protein tyrosine specific kinase is to be used.

The phosphoryl-group accepting amino acid position (S/T/Y/H/D/K) may be placed in a floating or fixed position in a peptide sequence motif. The floating position may be shifted towards either the amino-terminal or carboxyl-terminal in order to probe more stringently the specificity of the kinase in either terminus direction. It may be desirable to alter the position of the phosphoryl-group acceptor within a collection of peptides to maximize the diversity of the collection, or include multiple phosphoryl-accepting groups when screening for kinases that phosphorylate multiple neighboring amino acids.

Another approach to diversifying a peptide library is to dope one or more amino acid classes with either serine or threonine or one of the other phosphoryl-accepting amino acids. For example, by doping the binary mixture arginine and lysine with either serine or threonine, the binary mixture becomes a ternary mixture with two related basic amino acids (K and R) and one phosphate-accepting amino acid (S or T). Where this B position is doped with a phosphate-accepting amino acid, the position may be designated as B′. Doping different variable positions increases the library diversity.

The amino acid class bending (P) consists solely of proline (P), as indicated in Table 2. Proline is the most rigid of the twenty naturally occurring amino acids because its side chain is covalently linked with the main chain nitrogen. The resulting cyclic structure of proline is often found in the bends of folded protein chains and is not averse to being exposed to water. The cyclic structure and bending nature of proline make it a preferable choice for a variable position.

The peptide libraries of the present invention may comprise any number of peptides and may be designed according to the specific enzyme to be screened. For instance, a suitable substrate sequence for a particular enzyme might already be known, in which case the peptides of the present invention may be designed with only one or a few variable positions in order to facilitate the identification of peptide inhibitors. Depending on the length of the peptides employed, the size of the library will also vary.

Preferably, the libraries of the present invention are designed to be a manageable size wherein each individual peptide molecule or related mixture of molecules may be segregated into individual wells of a microtiter dish. Thus, the size of the libraries of the present invention is limited only by the number of microtiter dishes that may be manipulated and screened in a single experiment. For instance, a collection of about 1500 molecules would fit in a set of five 384-well plates, and could readily be screened in a single experiment. Similarly, a collection of about three thousand molecules could be readily screened using a set of ten 384-well plates. A library of 18,000 peptides could also be screened, for instance using a format of 16 peptides per well in twelve 96 well plates. Any size microtiter plate may be used, including 24-well, 96-well, 384-well plates, 1536-well plates, etc.

The peptide libraries of the invention may comprise peptides of any length. Preferably, the number of amino acids in each peptide molecule is no less than about three and no greater than about twenty-five. Peptides of about four to ten amino acids are particularly preferred for screening for kinase and phosphatase substrate sequences.

In some embodiments, the peptide molecules in the library are each associated with a detectable label, such as a fluorophore. Suitable fluorophores are selected from the group consisting of Bodipy, Texas Red, DAPI, Cy-Dyes, Lissamine, fluorescein, rhodamine, phycoerythrin, free or chelated lanthanide series salts and coumarin. Although, any detectable label may be employed, including colorimetric labels, luminescent labels, radiolabels, etc. Labels should be chosen with regard to charge characteristics as the charge on certain labels may affect the final pI of the peptidic substrate. The detectable label may be separated from amino acids of said peptide molecules by a suitable linker molecule as described in Application No. PCT/US02/02600, which is herein incorporated by reference in its entirety. Individual peptide molecules may also comprise a linker in the absence of a detectable label, as a linker provides the benefit of increasing the solubility of the peptide. Suitable linkers include polyethylene glycol (PEG) derivatives and polysaccharides having a molecular weight of about 80 to 4000 Daltons. In particular, Jeffamines are synthesized as either monoamines, diamines, or triamines, and are made in a variety of molecular weights ranging up to 5,000. Jeffamine ED-900, for instance, may be easily functionalized with a fluorophore and conjugated to a synthetic peptide.

Once designed, the peptides for the libraries of the present invention may be ordered from a commercial source. Alternatively, any method known in the art for synthesizing peptide sequences may be used to produce the peptides and libraries of the present invention. Ideally, peptides are synthesized directly in individually wells of a microtiter plate and their positions are pre-defined according to sequence.

Setting Peptide Library Constraints by Algorithm

As mentioned above, in some embodiments of the invention it may be helpful to use an algorithm to design a peptide library according to a specific set of constraints. Such an algorithm may be supplied on a computer readable medium containing computer executable instructions for defining the libraries of the invention. The peptide libraries of the invention contain peptides having the same charge or same like charge, so one constraint for a kinase library, for instance, would be for the number of basic residues (arginine or lysine) minus the number of acidic residues (aspartic acid or glutamic acid) to equal +1 (since the kinase adds a −2 charge to a suitable substrate). By reducing the number of amino acids from a specific class that can be present in any given peptide sequence, further constraints may be introduced. The more constraints used, the smaller the resulting library size. For instance, the number of peptides that are possible for a +1 library of 6mers after applying the following constraints—(# R)−(# E)=+1, # R<3, # L<3, and # P<2—reduces the library size from 15,625 (or 5⁶ members as discussed above) to 1,566.

For example, a computer program that satisfies the constraints listed above and generates a library of sequences according to the following formula is shown below:

Fluor-Xaa-Xaa-Xaa-Ser-Xaa-Xaa-Xaa-Gly-NH₂

The program generates all 15,625 sequences and culls out those that do not satisfy the constraints. The program listing is as follows:

```
#include <stdio.h>
int a[6];
void print_results(void){
      int j;
      printf(“J”);
      for(j=0;j<3;j++){
            switch (a[j]){
                  case 0:
                        printf(“R”);
                        break;
                  case 1:
                        printf(“E”);
                        break;
                  case 2:
                        printf(“P”);
                        break;
                  case 3:
                        printf(“L”);
                        break;
                  case 4:
                        printf(“A”);
                        break;
            }
      }
      printf(“S”);
      for(j=3;j<6;j++){
            switch (a[j]){
                  case 0:
                        printf(“R”);
                        break;
                  case 1:
                        printf(“E”);
                        break;
                  case 2:
                        printf(“P”);
                        break;
                  case 3:
                        printf(“L”);
                        break;
                  case 4:
                        printf(“A”);
                        break;
            }
      }
      printf(“G♯n”);
}
void main(void){
      int i = 0, ii=0, iii=0, iiii=0, iiiii=0;
      int i1,i2,i3,i4,i5,i6;
      int nL = 0;
      int nP = 0;
      int nC = 0;
      int nR = 0;
      int j;
      for (i1=0; i1<5; i1++){
            a[0] = i1;
// if(2 == i1){nP = 1;}else{nP = 0;}
            for (i2=0; i2<5; i2++){
                  a[1] = i2;
                  for (i3=0; i3<5; i3++){
                        a[2] = i3;
                        for (i4=0; i4<5; i4++){
                              a[3] = i4;
                              for (i5=0; i5<5; i5++){
                                    a[4] = i5;
                                    for (i6=0; i6<5; i6++){
                                          a[5] = i6;
                                          i++;
                                          nP = 0;
                                          for(j = 0; j<6; j++){
                                                if(a[j] == 2){nP++;}
                                          }
                                                if(nP < 2){ //np
                                                      ii++;
                                                      nL = 0;
                                                      for(j = 0; j<6; j++){
                                                            if(a[j] == 3){nL++;}
                                                      }
                                                      if(nL < 3){
                                                            iii++;
                                                            nC = 0;
                                                            for(j = 0; j<6; j++){
                                                                  if(a[j] ==
0){nC++;}
                                                                  if(a[j] == 1){nC−
−;}
                                                            }
                                                            if(nC == 1){
                                                                  iiii++;
                                                                  nR = 0;
                                                                  for(j = 0; j<6;
j++){
                                                                  if(a[j] ==
0){nR++;}
                                                            }
                                                            if(nR < 3){
            print_results( );
                                                                  iiiii++;
                                                            }
                                                      }
                                                }
                                          }
                                    }
                              }
                        }
                  }
            }
      }
// printf(“i = %d♯n”, i);
// printf(“ii = %d♯n”, ii);
// printf(“iii = %d♯n”, iii);
// printf(“iiii = %d♯n”, iiii);
// printf(“iiiii = %d♯n”, iiiii);
}
```

Screening Peptide Libraries: Exemplary Electrophoretic Apparatuses

The peptide libraries of the invention may be screened using any apparatus or system that permits identification of peptide substrates that have undergone a charge inversion when exposed to the enzyme of interest. Some embodiments will benefit from apparatuses permitting high throughput screening, such as those that permit testing hundreds or thousands of samples simultaneously.

Some exemplary systems which may be used to perform the methods of the invention include those described in Application Serial Nos. PCT/US01/43508, PCT/US01/44297, and PCT/US01/43504, which are each herein incorporated by reference in their entireties. For instance, Application Serial No. PCT/US01/43508, entitled “Microtiter Plate Format Device and Methods of Separating Differently Charged Molecules Using an Electric Field,” discloses a system comprising (i) a sample plate comprising a plurality of substantially tubular sample wells arrayed in the sample plate; (ii) at least one capture matrix, wherein the capture matrix is disposed in each of the sample wells proximate an end of the sample wells, and wherein the capture matrix comprises a diffusion-inhibiting material; (iii) at least one first electrode in electrical contact with at least one sample well at the bottom end of the sample well, and at least one second electrode in electrical contact with the top end of the sample well, wherein both electrodes are coupled to a power source. In general, such an apparatus includes a sample plate comprising a plurality of tubular sample wells, where each well contains a capture matrix designed to retain the molecule of interest upon electrophoresis of a sample. The system also contains at least one pair of electrodes. Each discrete sample well is in electrical contact with a first electrode near the bottom of the well, and a second electrode near the top of the well.

In the microtiter apparatuses described in Application Serial No. PCT/US01/43508, the capture matrix comprises a diffusion-inhibiting material that retards the free diffusion of molecules. This material serves two functions: first, to ensure that the charged molecules of interest are retained for detection within the capture matrix after electrophoresis; and second, to prevent other molecules from diffusing into the capture matrix. The capture matrix preferably also contains other layers of material which bind the charged molecules of interest. Such a binding layer captures the charged molecule of interest in a specific or non-specific manner in order to hold the charged molecules of interest in a particular location for detection, which allows more facile quantification of the molecule of interest as compared to a diffusion-inhibiting layer only capture matrix. As the binding layer will often also bind other molecules in the sample, the second function of the diffusion-inhibiting material is important in these embodiments.

For use of the microtiter plates with the libraries of the present invention, individual peptides are loaded (or have been pre-loaded) into the wells of the sample plate. The peptides are then exposed to the enzyme in a suitable buffer and with reactants required to detect enzyme activity. The peptides are then electrophoresed in a liquid which supports the electrophoretic movement of the peptides, preferably an aqueous buffer. Upon electrophoresis, peptides having undergone a charge inversion are selectively transported and concentrated in the capture matrix. The molecules with a negative charge move towards the anode and may be sequestered by a capture matrix placed between the sample and the anode. Alternately, molecules with a positive charge move towards the cathode and may be sequestered by a capture matrix placed between the sample and the cathode. Uncharged molecules, and those of a charge not captured by the capture matrix, are washed out of the sample wells and apparatus with a washing buffer. Alternatively, molecules of an undesired charge are electrophoretically moved into one of the buffer reservoirs of the apparatus, where they may be removed by continuously replenishing the buffer. The peptides that are retained in the capture matrix may then be detected by any appropriate means, including fluorometry, colorimetry, luminometry, mass spectrometry, electrochemical detection, and radioactivity detection.

Application Serial No. PCT/US01/43504, entitled “Microstructure Apparatus and Method for Separating Differently Charged Molecules Using an Applied Electric Field,” discloses a system comprising (i) a microstructure plate comprising at least one microstructure, each microstructure comprising a series of microstructure sections and channels, wherein each microstructure section is directly interconnected to at least one other microstructure section by at least one channel, the series comprising at least one sample accepting microstructure section, wherein the sample accepting section is fluidly connected to the exterior of the microstructure plate; at least one first electrode microstructure section; at least one second electrode microstructure section; at least one capture microstructure section containing a capture matrix, wherein the capture microstructure section is between the first and second electrode microstructure sections in the series; wherein the microstructures in the microstructure plate are formed by at least two layers of material, wherein at least one layer is a sealing plate layer which seals at least one channel or microstructure section in the assembled microstructure plate; and (ii) an electrode assembly, the electrode assembly having at least one first and at least one second electrode, wherein each first electrode microstructure section is in electrical contact with at least one first electrode, and wherein each second electrode microstructure section is in electrical contact with at least one second electrode.

In general, the microstructure plate disclosed in Application Serial No. PCT/US01/43504 is a laminar structure which forms the set of microstructures. Each microstructure comprises a set of microstructure sections (defined by function) and channels connecting those sections. The microstructures include at least one sample accepting microstructure section, which is fluidly connected to the exterior of the microstructure plate. Usually this fluid connection is accomplished by an opening in one or more layers of the microstructure plate. The microstructures also include at least one first electrode microstructure section and at least one second electrode microstructure section. These sections are either adapted to accept an electrode (e.g., have openings to the exterior of the microstructure plate which permit the entry of pin electrodes), or contain electrodes (e.g., integrally molded electrodes).

The microstructures of Application Serial No. PCT/US01/43504 may also include at least one capture microstructure section containing a capture matrix. This capture microstructure section, which is between the first and second electrode microstructure sections in the series, binds or holds the charged molecule of interest when a sample is electrophoresed, so that the molecule may later be detected. Openings to the exterior of the microstructure plate may be formed in any layer of the microstructure plate for the injection of samples, access of electrodes, or for optical access to the capture chamber, may be present in various embodiments of the system.

Application Serial No. PCT/US01/44297, entitled “Microcapillary Arrays for High-Throughput Screening and Separation of Differently Charged Molecules Using an Electric Field,” discloses a system microtiter plate comprising a plurality of first and second wells, wherein (i) said first and second wells contain a liquid and are connected by a capillary tube fluid circuit which is also filled with a liquid; (ii) said capillary tube fluid circuit comprises a detection section in which passage of a reaction product may be detected; (iii) said first and second wells are in contact with first and second electrodes for applying an electric current across the capillary tube fluid circuit; and (iv) said electrodes are connected to a power supply. In general, the systems disclosed in this application comprise at least one separatory unit, although systems with a plurality of separatory units are preferred. Each separatory unit consists of a first and second well on the same microtiter plate connected by a capillary tube fluid circuit, and a first and second electrode electrically connected to the first and second well.

The capillary tube fluid circuit has a length sufficient to allow electrophoretic separation of molecular analytes, and a cross section small enough to allow the application of high electric fields without excessive power consumption, but large enough to allow filling and cleaning of the circuit with moderate pressure. The capillary tube fluid circuit also comprises a section through which a molecule of interest may be detected (e.g., a transparent section), which may be either specially fabricated or may take advantage of an intrinsic property of the capillary tube material.

The fluid circuit, and the two wells, contain a liquid which promotes electro-kinetic transport of molecular analytes when an electric field is applied. The liquid may also flow electro-osmotically under the applied electric field. In operation, a sample is contained within the first well. The separatory unit is briefly energized in order to draw an amount of the sample into the capillary tube fluid circuit. The fluid circuit is then moved to a third and fourth well (the new first and second wells of the separatory unit) containing the liquid, which are in contact with a third and fourth electrode, and the system is again energized. Molecules are transported through the fluidic circuit by electro-osmotic flow and/or electrophoretic means, with the molecules of greatest electrophoretic mobility in the direction of the fourth electrode moving at the greatest rate. The separated molecules are detected as they flow through the detection section of the fluid circuit by a detection device, such as fluorometer.

Other appropriate apparatuses for screening the libraries of the present invention are known in the art. For instance, enzyme reaction mixtures may be applied to a planar agarose gel and exposed to an electric current, and the labeled peptides having undergone charge inversion due to activity of the enzyme may be visualized after migrating towards the oppositely charged electrode in the gel. One exemplary gel system that could be used is the 2% E-Gel® 96 Agarose gel sold by Invitrogen, which is a bufferless, pre-cast agarose gel designed for fast, high-throughput DNA electrophoresis. Each gel contains 96 sample lanes and 8 marker lanes with a 96-well staggered-well layout providing a 1.6 cm run length. The E-Gel® 96 loading format is compatible with multi-channel pipettors, and the most commonly used 8-, 12-, and 96-pin liquid handling robots. With just 12-minute run times, up to 20,000 samples can be resolved in a single day. Another exemplary planar gel system is the MADGE (Microplate Array Diagonal Gel Electrophoresis) Bio Gel. A 96-well manual pipettor may be used for the gels, which are fully microplate compatible for 8×12 or 96 channel air displacement pipetting or passive transfer. With short run times, hundreds or thousands of samples can be screened daily.

Kinase Screening

The peptide libraries described herein are useful for screening for kinase substrate activity. The substrate specificity for any protein kinase may be tested using the disclosed libraries, including kinases of prokaryotic, eukaryotic, bacterial, viral, fungal or archaea origin. The disclosed libraries are particularly useful for screening potential substrates for tyrosine, serine/threonine or histidine protein kinases. Specific examples of kinases that may be screened include, but are not limited to, LCK, IRK (=INSR=Insulin receptor), IGF-1 receptor, SYK, ZAP-70, IRAK1, IRAK2, BLK, BMX, BTK, FRK, FGR, FYN, HCK, ITK, LYN, TEC, TXK, YES, ABL, SRC, EGF-R (=ErbB-1), ErbB-2 (=NEU=HER2), ErbB-3, ErbB-4, FAK, FGF1R (=FGR-1), FGF2R (=FGR-2), IKK-1 (=IKK-ALPHA=CHUK), IKK-2 (=IKK-BETA), MET (=c-MET), NIK, PDGF receptor ALPHA, PDGF receptor BETA, TIE1, TIE2 (=TEK), VEGFR1 (=FLT-1), VEGFR2 (=KDR), FLT-3, FLT4, KIT, CSK, JAK1, JAK2, JAK3, TYK2, RIP, RIP-2, LOK, TAK1, RET, ALK, MLK3, COT, TRKA, PYK2, EPHB4, RON, GSK3, UL13, ORF47, ATM, CDK (including all subtypes), PKA, PKB (including all PKB subtypes) (=AKT-1, AKT-2, AKT-3), PKC (including all PKC subtypes), and bARK1 (=GRK2) (and other G-protein coupled receptor kinases (GRKs)), and all subtypes of these kinases. Additional kinases are listed in Manning et al., 2002, Science 298: 1912, which is herein incorporated by reference in its entirety.

As mentioned above, the libraries of the invention may be designed by starting with known substrate sequences and altering the amino acids around the phosphorylation site to develop improved substrates or inhibitors. For this purpose, any known substrate sequence may be used, and will vary depending on the kinase of interest. Some exemplary substrate sequences for SRC kinase and protein kinase A, for example, are disclosed in PCT/US02/02600, which is herein incorporated by reference in its entirety. Exemplary substrate sequences have also been identified for casein kinases I and II, NIMA, phosphorylase kinase, calmodulin-dependent kinase II, CDK5, and Erk1 (Songyang et al., 1996, Mol. Cell. Biol. 16(11): 6486-93), protein kinase C (Nishikawa et al., 1997, J. Biol. Chem. 272(2): 952-60), and protein kinase B (Obata et al., 2000, J. Biol. Chem. 275(46): 36108-15), to name a few. See also Kemp and Pearson, 1991, Design and use of peptide substrates for protein kinases, Methods Enzymol. 200: 121-35, and Pearson and Kemp, 1991, Protein kinase phosphorylation site sequences and consensus specificity motifs: tabulations, Methods Enzymol. 200: 62-81, each of which is incorproated by reference in its entirety. There is also a database called PhosphoBase, which contains a listing of experimentally verified phosphorylation sites and associated protein kinases. The data are collected from literature and the SwissProt database and stored in a relational SQL database. Version 3.0 contains 1766 phosphorylated residues with 1310 protein kinase annotations. PhosphoBase is available on the World Wide Web from the CBS Server at http://www.cbs.dtu.dk/databases/PhosphoBase.

Phosphatase Screening

The peptide libraries described herein are also useful for screening for phosphatase substrate activity. The substrate specificity for any protein phosphatase may be tested using the disclosed libraries, including phosphatases of prokaryotic, eukaryotic, bacterial, viral, fungal or archaea origin. The disclosed libraries are particularly useful for screening potential substrates for tyrosine, serine/threonine or histidine protein phosphatases. Specific examples of tyrosine phosphatases that may be screened include, but are not limited to, SHP-1, SHP-2, PTP1B, PTPMEG, PTP1c, Yop51, VH1, cdc25, CD45, HLAR, PTP18, HPTPalpha, MKPs and DPTP10D. Other tyrosine phosphatases are disclosed in Mustelin et al., 2002, Frontiers in Bioscience 7: d85-142, and Li and Dixon, 2000, Sem. Immunol. 12(1): 75-84, each of which is herein incorporated by reference in its entirety. Specific serine-threonine phosphatases include, but are not limited to, any of the enzymes encoded by the PPP-gene family, including PP1, PP2A, PP2B, PP4, PP5, PP6 and PP7. The use of phosphatases that counter the substrate specificity of the specific kinases listed above is also contemplated.

As mentioned above, the libraries of the invention may be designed by starting with known substrate sequences and altering the amino acids around the dephosphorylation site to develop improved substrates or inhibitors. For this purpose, any known substrate sequence may be used, and will vary depending on the phosphatase of interest. Some exemplary substrate sequences for PTP1B and MKP3, for example, are disclosed in Zhang, 2002, Annu. Rev. Pharmacol. Toxicol. 42:209-34, which is herein incorporated by reference in its entirety. See also Theodosiou and Ashworth, 2002, MAP kinase phosphatases, Genome Biol. 3(7): REVIEWS3009, and Zhang and Dixon, 1994, Protein tyrosine phosphatases: mechanism of catalysis and substrate specificity, Adv. Enzymol. Relat. Areas Mol. Biol. 68: 1-36, each of which is incorporated by reference in its entirety.

Other Utilities

The libraries of the present invention are particularly suitable for screening for substrates that undergo a change in net charge upon exposure to an enzyme, for instance, a kinase or phosphatase enzyme, which each transfer a phosphoryl group (PO₃²⁻). However, the libraries of the present invention may also be used to identify sequence motifs involved in other types of ion exchange or transfer events, for instance those involving +1 ions such as hydrogen (H⁺), sodium (Na⁺), potassium (K⁺), mercury (Hg⁺), ammonium (NH⁺), silver (Ag⁺) and cuprous (Cu⁺); −1 ions such as fluoride (F⁻), chloride (Cl⁻), hydroxide (OH⁻) and nitrate (NO³⁻); +2 ions such as calcium (Ca²⁺), magnesium (Mg²⁺), barium (Ba²⁺), cupric (Cu²⁺), zinc (Zn²⁺), mercuric (Hg²⁺) and ferrous (Fe²⁺); −2 ions such as oxide (O²⁻), sulfide (S²⁻), sulfite (SO₃²⁻), sulfate (SO₄²⁻) and carbonate (CO₃²⁻); +3 ions such as aluminum (Al³⁺) and ferric (Fe³⁺); and −3 ions such as phosphate (PO₄³⁻).

For instance, the peptide libraries of the invention may be used to screen for peptides that have high affinities for toxic metals. Such screening may allow for the systematic identification of peptides useful in chelation therapy, as well as for the identification of metal-binding sites in other types of proteins. The libraries of the invention may also be used to identify or verify cation binding sites in ion transporting ATPases (see Ogawa and Toyoshima, 2002, Proc. Natl. Acad. Sci. USA 99(25): 15977-82).

The libraries of the invention may also be designed to identify sequences of effector proteins or peptides that activate, promote, enhance or facilitate phosphorylation or dephosphorylation. For instance, such a kinase screening assay could be performed with two peptide components: 1) a fluorophor- or other labeled peptide containing a potential phosphorylation site with a net (+1) charge where the same sequence is used in each screening reaction, and 2) a peptide “effector” library that is unlabeled but is present in different wells of a microtiter plate, as single peptides or motifs of peptides, i.e., binary or other groupings. In the absence of an effector, the fluorophore-labeled phosphorylation-site peptide would not be phosphoylated by the kinase, or would be phosphorylated at only a low level. The purpose would be to identify which peptide effector sequences or motifs bind to the kinase or to the peptide and activate phosphorylation of the phosphorylation-site containing labeled peptide, or that contain neighboring or conformational sequences from the substrate protein that facilitate phosphorylation.

Thus, for the screening of a kinase effector peptide library, a library may be designed with two peptides, a labeled (+1) peptide substrate and an effector peptide sequence present in the reaction. Alternatively, the sequence/motif from the effector library screen could be combined into a single effector/phosphorylation site labeled (+1) peptide in the design of the library.

In instances where the libraries are used to identify enzyme substrates, the substrates identified may be used to design assays to detect or monitor enzyme activity. For instance, such assays are described in copending application Ser. No. 60/413,137, which is herein incorporated by reference in its entirety. The amino acid sequence of substrates identified according to the methods of the present invention may also be altered to produce better substrates, or to produce enzyme inhibitors. Such inhibitors may be used to treat any disease associated with the particular enzyme of interest, either alone or in combination with other therapeutic compounds.

For instance, several highly selective pseudosubstrate-based peptide inhibitors have already been developed for certain protein tyrosine kinases in an effort to develop inhibitors for diseases such as cancer, psoriasis, and osteoporosis. al-Obeidi et al., 1998, Biopolymers 47(3): 197-223. Mitogen-activated protein kinase, which is involved in the regulation of cell proliferation and differentiation, has been selectively inhibited using cell-permeable peptide inhibitors fused to either an alkyl moiety or a membrane-translocating peptide sequence. Kelemen et al., 2002, J. Biol. Chem. 277(10): 8741-8. And peptides containing the non-hydrolysable phosphotyrosine analogue 4-[difluro(phosphono)methyl]phenylalanine [Phe(CF2P)] have been shown to be potent inhibitors of phosphotyrosine phosphatases. Desmarais et al., 1999, Biochem. J. 337 (Pt 2): 219-23. Thus, the peptide substrates identified for kinases and phosphatases using the methods and libraries of the present invention may be used to design peptide inhibitors by making known modifications to the peptide backbone, including modifications that permit cell membrane translocation and chemical modifications that create inhibition in the absence of sequence changes.

### EXAMPLES

### Example 1

As described above, an algorithm may be employed to generate the kinase peptide libraries of the present invention using the constraint (#B)−(#Z)=+1. To limit the number of members in the library, the algorithm employed may contain additional constraints to further reduce the number of peptides in the library. For instance, the number of peptides that are possible for a +1 library after applying the following constraints—(# R)−(# E)=+1, # R<3, # L<3, and # P<2—reduces the library size from 15,625 (or 5⁶ members as discussed above) to 1,566. See Table 5 below for an exemplary library of peptides generated using such an algorithm, using the amino acids arginine (R), glutamic acid (E), alanine (A), leucine (L), and proline (P), with a serine residue fixed in the center position. Note that in this library, “J” denotes a fluorophore-modified amino acid rather than a spacing amino acid. The glycine residue at the termini of the peptide sequences facilitates solid-phase synthesis.

TABLE 5
Exemplary Peptide Library

| | | | | | | | |
|---|---|---|---|---|---|---|---|
| JRRESPLLG | JPAESRRLG | JRALSEPRG | JARESARAG | JRPASERLG | JLPESRRLG | JELASRLRG | JALRSRAEG |
| JRRESPLAG | JPAESRRAG | JRALSELRG | JARESAPRG | JRPASERAG | JLPESRRAG | JELASRARG | JALRSERPG |
| JRRESPALG | JPAESRLRG | JRALSEARG | JARESALRG | JRPASELRG | JLPESRLRG | JELASPRRG | JALRSERLG |
| JRRESPAAG | JPAESRARG | JRALSPREG | JARESAARG | JRPASEARG | JLPESRARG | JELASLRRG | JALRSERAG |
| JRRESLPLG | JPAESLRRG | JRALSPERG | JARPSRELG | JRPASLREG | JLPESLRRG | JELASARRG | JALRSEPRG |
| JRRESLPAG | JPAESARRG | JRALSPLAG | JARPSREAG | JRPASLERG | JLPESARRG | JEARSRPLG | JALRSELRG |
| JRRESLLPG | JPALSRREG | JRALSPALG | JARPSRLEG | JRPASLLAG | JLPLSRREG | JEARSRPAG | JALRSEARG |
| JRRESLLAG | JPALSRERG | JRALSPAAG | JARPSRAEG | JRPASLALG | JLPLSRERG | JEARSRLPG | JALRSPREG |
| JRRESLAPG | JPALSRLAG | JRALSLREG | JARPSERLG | JRPASLAAG | JLPLSRAAG | JEARSRLLG | JALRSPERG |
| JRRESLALG | JPALSRALG | JRALSLERG | JARPSERAG | JRPASAREG | JLPLSERRG | JEARSRLAG | JALRSPLAG |
| JRRESLAAG | JPALSRAAG | JRALSLPAG | JARPSELRG | JRPASAERG | JLPLSARAG | JEARSRAPG | JALRSPALG |
| JRRESAPLG | JPALSERRG | JRALSLAPG | JARPSEARG | JRPASALLG | JLPLSAARG | JEARSRALG | JALRSPPAG |
| JRRESAPAG | JPALSLRAG | JRALSLAAG | JARPSLREG | JRPASALAG | JLPASRREG | JEARSRAAG | JALRSLREG |
| JRRESALPG | JPALSLARG | JRALSAREG | JARPSLERG | JRPASAALG | JLPASRERG | JEARSPRLG | JALRSLERG |
| JRRESALLG | JPALSARLG | JRALSAERG | JARPSLLAG | JRPASAAAG | JLPASRLAG | JEARSPRAG | JALRSLPAG |
| JRRESALAG | JPALSARAG | JRALSAPLG | JARPSLALG | JRLRSEPLG | JLPASRALG | JEARSPLRG | JALRSLAPG |
| JRRESAAPG | JPALSALRG | JRALSAPAG | JARPSLAAG | JRLRSEPAG | JLPASRAAG | JEARSPARG | JALRSLAAG |
| JRRESAALG | JPALSAARG | JRALSALPG | JARPSAREG | JRLRSELPG | JLPASERRG | JEARSLRPG | JALRSAREG |
| JRRESAAAG | JPAASRREG | JRALSALAG | JARPSAERG | JRLRSELAG | JLPASLRAG | JEARSLRLG | JALRSAERG |
| JRRPSELLG | JPAASRERG | JRALSAAPG | JARPSALLG | JRLRSEAPG | JLPASLARG | JEARSLRAG | JALRSAPLG |
| JRRPSELAG | JPAASRLLG | JRALSAALG | JARPSALAG | JRLRSEALG | JLPASARLG | JEARSLPRG | JALRSAPAG |
| JRRPSEALG | JPAASRLAG | JRALSAAAG | JARPSAALG | JRLRSEAAG | JLPASARAG | JEARSLLRG | JALRSALPG |
| JRRPSEAAG | JPPASRALG | JRAASREPG | JARPSAAAG | JRLRSPELG | JLPASALRG | JEARSLARG | JALRSALAG |
| JRRPSLELG | JPAASRAAG | JRAASRELG | JARLSREPG | JRLRSPEAG | JLPASAARG | JEARSARPG | JALRSAAPG |
| JRRPSLEAG | JPAASERRG | JRAASREAG | JARLSRELG | JRLRSPLEG | JLLRSREPG | JEARSARLG | JALRSAALG |
| JRRPSLLEG | JPAASLRLG | JRAASRPEG | JARLSREAG | JRLRSPAEG | JLLRSREAG | JEARSARAG | JALRSPAAG |
| JRRPSLAEG | JPAASLRAG | JRAASRLEG | JARLSRPEG | JRLRSLEPG | JLLRSRPEG | JEARSAPRG | JALESRRPG |
| JRRPSAELG | JPAASLLRG | JRAASRAEG | JARLSRLEG | JRLRSLEAG | JLLRSRAEG | JEARSALRG | JALESRRLG |
| JRRPSAEAG | JPAASLARG | JRAASERPG | JARLSRAEG | JRLRSLPEG | JLLRSERPG | JEARSAARG | JALESRRAG |
| JRRPSALEG | JPAASARLG | JRAASERLG | JARLSERPG | JRLRSLAEG | JLLRSERAG | JEAPSRRLG | JALESRPRG |
| JRRPSAAEG | JPAASARAG | JRAASERAG | JARLSERLG | JRLRSAEPG | JLLRSEPRG | JEAPSRRAG | JALESRLRG |
| JRRLSEPLG | JPAASALRG | JRAASEPRG | JARLSERAG | JRLRSAELG | JLLRSEARG | JEAPSRLRG | JALESRARG |
| JRRLSEPAG | JPAASPARG | JRAASELRG | JARLSEPRG | JRLRSAEAG | JLLRSPREG | JEAPSRARG | JALESPRRG |
| JRRLSELPG | JLRRSEPLG | JRAASEARG | JARLSELRG | JRLRSAPEG | JLLRSPERG | JEAPSLRRG | JALESLRRG |
| JRRLSELAG | JLRRSEPAG | JRAASPREG | JARLSEARG | JRLRSALEG | JLLRSPAAG | JEAPSARRG | JALESARRG |
| JRRLSEAPG | JLRRSELPG | JRAASPERG | JARLSPREG | JRLRSAAEG | JLLRSAREG | JEALSRRPG | JALPSRREG |
| JRRLSEALG | JLRRSELAG | JRAASPLLG | JARLSPERG | JRLESRPLG | JLLRSAERG | JEALSRRLG | JALPSRERG |
| JRRLSEAAG | JLRRSEAPG | JRAASPLAG | JARLSPLAG | JRLESRPAG | JLLRSAPAG | JEALSRRAG | JALPSRLAG |
| JRRLSPELG | JLRRSEALG | JRAASPALG | JARLSPALG | JRLESRLPG | JLLRSAAPG | JEALSRPRG | JALPSRALG |
| JRRLSPEAG | JLRRSEAAG | JRAASPAAG | JARLSPAAG | JRLESRLAG | JLLRSAAAG | JEALSRLRG | JALPSRAAG |
| JRRLSPLEG | JLRRSPELG | JRAASLREG | JARLSLREG | JRLESRAPG | JLLESRRPG | JEALSRARG | JALPSERRG |
| JRRLSPAEG | JLRRSPEAG | JRAASLERG | JARLSLERG | JRLESRALG | JLLESRRAG | JEALSPRRG | JALPSLRAG |
| JRRLSLEPG | JLRRSPLEG | JRAASLPLG | JARLSLPAG | JRLESRAAG | JLLESRPRG | JEALSLRRG | JALPSLARG |
| JRRLSLEAG | JLRRSPAEG | JRAASLPAG | JARLSLAPG | JRLESPRLG | JLLESRARG | JEALSARRG | JALPSARLG |
| JRRLSLPEG | JLRRSLEPG | JRAASLLPG | JARLSLAAG | JRLESPRAG | JLLESPRRG | JEAASRRPG | JALPSARAG |
| JRRLSLAEG | JLRRSLEAG | JRAASLLAG | JARLSAREG | JRLESPLRG | JLLESARRG | JEAASRRLG | JALPSALRG |
| JRRLSAEPG | JLRRSLPEG | JRAASLAPG | JARLSAERG | JRLESPARG | JLLPSRREG | JEAASRRAG | JALPSAARG |
| JRRLSAELG | JLRRSLAEG | JRAASLALG | JARLSAPLG | JRLESLRPG | JLLPSRERG | JEAASRPRG | JALLSRREG |
| JRRLSAEAG | JLRRSAEPG | JRAASLAAG | JARLSAPAG | JRLESLRAG | JLLPSRAAG | JEAASRLRG | JALLSRERG |
| JRRLSAPEG | JLRRSAELG | JRAASAREG | JARLSALPG | JRLESLPRG | JLLPSERRG | JEAASRARG | JALLSRPAG |
| JRRLSALEG | JLRRSAEAG | JRAASAERG | JARLSALAG | JRLESLARG | JLLPSARAG | JEAASPRRG | JALLSRAPG |
| JRRLSAAEG | JLRRSAPEG | JRAASAPLG | JARLSAAPG | JRLESARPG | JLLPSAARG | JEAASLRRG | JALLSRAAG |
| JRRASEPLG | JLRRSALEG | JRAASAPAG | JARLSAALG | JRLESARLG | JLLASRREG | JEAASARRG | JALLSERRG |
| JRRASEPAG | JLRRSAAEG | JRAASALPG | JARLSAAAG | JRLESARAG | JLLASRERG | JPRRSELLG | JALLSPRAG |
| JRRASELPG | JLRESRPLG | JRAASALLG | JARASREPG | JRLESAPRG | JLLASRPAG | JPRRSELAG | JALLSPARG |
| JRRASELLG | JLRESRPAG | JRAASALAG | JARASRELG | JRLESALRG | JLLASRAPG | JPRRSEALG | JALLSARPG |
| JRRASELAG | JLRESRLPG | JRAASAAPG | JARASREAG | JRLESAARG | JLLASRAAG | JPRRSEPAG | JALLSARAG |
| JRRASEAPG | JLRESRLAG | JRAASAALG | JARASRPEG | JRLPSRELG | JLLASERRG | JPRRSLELG | JALLSAPRG |
| JRRASEALG | JLRESRAPG | JRAASAAAG | JARASRLEG | JRLPSREAG | JLLASPRAG | JPRRSLEAG | JALLSAARG |
| JRRASEAAG | JLRESRALG | JERRSPLLG | JARASRAEG | JRLPSRLEG | JLLASPARG | JPRRSLLEG | JALASRREG |
| JRRASPELG | JLRESRPAG | JERRSPLAG | JARASERPG | JRLPSRAEG | JLLASARPG | JPRRSLAEG | JALASRERG |
| JRRASPEAG | JLRESPRLG | JERRSPALG | JARASERLG | JRLPSERLG | JLLASARAG | JPRRSAELG | JALASRPLG |
| JRRASPLEG | JLRESPRAG | JERRSPAAG | JARASERAG | JRLPSERAG | JLLASAPRG | JPRRSAEAG | JALASRPAG |
| JRRASPAEG | JLRESPLRG | JERRSLPLG | JARASEPRG | JRLPSELRG | JLLASAARG | JPRRSALEG | JALASRLPG |
| JRRASLEPG | JLRESPARG | JERRSLPAG | JARASELRG | JRLPSEARG | JLARSREPG | JPRRSAAEG | JALASRLAG |
| JRRASLELG | JLRESLRPG | JERRSLLPG | JARASEARG | JRLPSLREG | JLARSRELG | JPRESRLLG | JALASRAPG |
| JRRASLEAG | JLRESLRAG | JERRSLLAG | JARASPREG | JRLPSLERG | JLARSREAG | JPRESRLAG | JALASRALG |
| JRRASLPEG | JLRESLPRG | JERRSLAPG | JARASPERG | JRLPSLAAG | JLARSRPEG | JPRESRALG | JALASRAAG |
| JRRASLLEG | JLRESLARG | JERRSLALG | JARASPLLG | JRLPSAREG | JLARSRLEG | JPRESRPAG | JALASERRG |
| JRRASLAEG | JLRESARPG | JERRSLAAG | JARASPLAG | JRLPSAERG | JLARSRAEG | JPRESLRLG | JALASPRLG |
| JRRASAEPG | JLRESARLG | JERRSAPLG | JARASPALG | JRLPSALAG | JLARSERPG | JPRESLRAG | JALASPRAG |
| JRRASAELG | JLRESARAG | JERRSAPAG | JARASPAAG | JRLPSAALG | JLARSERLG | JPRESLLRG | JALASPLRG |
| JRRASAEAG | JLRESAPRG | JERRSALPG | JARASLREG | JRLPSAAAG | JLARSERAG | JPRESLARG | JALASPARG |
| JRRASAPEG | JLRESALRG | JERRSALLG | JARASLERG | JRLLSREPG | JLARSEPRG | JPRESARLG | JALASLRPG |
| JRRASALEG | JLRESAARG | JERRSALAG | JARASLPLG | JRLLSREAG | JLARSELRG | JPRESARAG | JALASLRAG |
| JRRASAAEG | JLRPSRELG | JERRSAAPG | JARASLPAG | JRLLSRPEG | JLARSEARG | JPRESALRG | JALASLPRG |
| JRERSPLLG | JLRPSREAG | JERRSAALG | JARASLLPG | JRLLSRAEG | JLARSPREG | JPRESAARG | JALASLARG |
| JRERSPLAG | JLRPSRLEG | JERRSAAAG | JARASLLAG | JRLLSERPG | JLARSPERG | JPRLSRELG | JALASARPG |
| JRERSPALG | JLRPSRAEG | JERPSRLLG | JARASLAPG | JRLLSERAG | JLARSPLAG | JPRLSREAG | JALASARLG |
| JRERSPAAG | JLRPSERLG | JERPSRLAG | JARASLALG | JRLLSEPRG | JLARSPALG | JPRLSRLEG | JALASARAG |
| JRERSLPLG | JLRPSERAG | JERPSRALG | JARASLAAG | JRLLSEARG | JLARSPAAG | JPRLSRAEG | JALASAPRG |
| JRERSLPAG | JLRPSELRG | JERPSRAAG | JARASAREG | JRLLSPREG | JLARSLREG | JPRLSERLG | JALASALRG |
| JRERSLLPG | JLRPSEARG | JERPSLRLG | JARASAERG | JRLLSPERG | JLARSLERG | JPRLSERAG | JALASAARG |
| JRERSLLAG | JLRPSLREG | JERPSLRAG | JARASAPLG | JRLLSPAAG | JLARSLPAG | JPRLSELRG | JAARSREPG |
| JRERSLAPG | JLRPSLERG | JERPSLLRG | JARASAPAG | JRLLSAREG | JLARSLAPG | JPRLSEARG | JAARSRELG |
| JRERSLALG | JLRPSLAAG | JERPSLARG | JARASALPG | JRLLSAERG | JLARSLAAG | JPRLSLREG | JAARSREAG |
| JRERSLAAG | JLRPSAREG | JERPSARLG | JARASALLG | JRLLSAPAG | JLARSAREG | JPRLSLERG | JAARSRPEG |
| JRERSAPLG | JLRPSAERG | JERPSARAG | JARASALAG | JRLLSAAPG | JLARSAERG | JPRLSLAAG | JAARSRLEG |
| JRERSAPAG | JLRPSALAG | JERPSALRG | JARASAAPG | JRLLSAAAG | JLARSAPLG | JPRLSAREG | JAARSRAEG |
| JRERSALPG | JLRPSAALG | JERPSAARG | JARASAALG | JRLASREPG | JLARSAPAG | JPRLSAERG | JAARSERPG |
| JRERSALLG | JLRPSAAAG | JERLSRPLG | JARASAAAG | JRLASRELG | JLARSALPG | JPRLSALAG | JAARSERLG |
| JRERSALAG | JLRLSREPG | JERLSRPAG | JAERSRPLG | JRLASREAG | JLARSALAG | JPRLSAALG | JAARSERAG |
| JRERSAAPG | JLRLSREAG | JERLSRLPG | JAERSRPAG | JRLASRPEG | JLARSAAPG | JPRLSAAAG | JAARSEPRG |
| JRERSAALG | JLRLSRPEG | JERLSRLAG | JAERSRLPG | JRLASRLEG | JLARSAALG | JPRASRELG | JAARSELRG |
| JRERSAAAG | JLRLSRAEG | JERLSRAPG | JAERSRLLG | JRLASRAEG | JLARSAAAG | JPRASREAG | JAARSEARG |
| JREPSRLLG | JLRLSERPG | JERLSRALG | JAERSRLAG | JRLASERPG | JLAESRRPG | JPRASRLEG | JAARSPREG |
| JREPSRLAG | JLRLSERAG | JERLSRAAG | JAERSRAPG | JRLASERLG | JLAESRRLG | JPRASRAEG | JAARSPERG |
| JREPSRALG | JLRLSEPRG | JERLSPRLG | JAERSRALG | JRLASERAG | JLAESRRAG | JPRASERLG | JAARSPLLG |
| JREPSRAAG | JLRLSEARG | JERLSPRAG | JAERSRAAG | JRLASEPRG | JLAESRPRG | JPRASERAG | JAARSPLAG |
| JREPSLRLG | JLRLSPREG | JERLSPLRG | JAERSPRLG | JRLASELRG | JLAESRLRG | JPRASELRG | JAARSPALG |
| JREPSLRAG | JLRLSPERG | JERLSPARG | JAERSPRAG | JRLASEARG | JLAESRARG | JPRASEARG | JAARSPAAG |
| JREPSLLRG | JLRLSPAAG | JERLSLRPG | JAERSPLRG | JRLASPREG | JLAESPRRG | JPRASLREG | JAARSLREG |
| JREPSLARG | JLRLSAREG | JERLSLRAG | JAERSPARG | JRLASPERG | JLAESLRRG | JPRASLERG | JAARSLERG |
| JREPSARLG | JLRLSAERG | JERLSLPRG | JAERSLRPG | JRLASPLAG | JLAESARRG | JPRASLLAG | JAARSLPLG |
| JREPSARAG | JLRLSAPAG | JERLSLARG | JAERSLRLG | JRLASPALG | JLAPSRREG | JPRASLALG | JAARSLPAG |
| JREPSALRG | JLRLSAAPG | JERLSARPG | JAERSLRAG | JRLASPAAG | JLAPSRERG | JPRASLAAG | JAARSLLPG |
| JREPSAARG | JLRLSAAAG | JERLSARLG | JAERSLPRG | JRLASLREG | JLAPSRLAG | JPRASAREG | JAARSLLAG |
| JRELSRPLG | JLRASREPG | JERLSARAG | JAERSLLRG | JRLASLERG | JLAPSRALG | JPRASAERG | JAARSLAPG |
| JRELSRPAG | JLRASRELG | JERLSAPRG | JAERSLARG | JRLASLPAG | JLAPSRAAG | JPRASALLG | JAARSLALG |
| JRELSRLPG | JLRASREAG | JERLSALRG | JAERSARPG | JRLASLAPG | JLAPSERRG | JPRASALAG | JAARSLAAG |
| JRELSRLAG | JLRASRPEG | JERLSAARG | JAERSARLG | JRLASLAAG | JLAPSLRAG | JPRASAALG | JAARSAREG |
| JRELSRAPG | JLRASRLEG | JERASRPLG | JAERSARAG | JRLASAREG | JLAPSLARG | JPRASAAAG | JAARSAERG |
| JRELSRALG | JLRASRAEG | JERASRPAG | JAERSAPRG | JRLASAERG | JLAPSARLG | JPERSRLLG | JAARSAPLG |
| JRELSRAAG | JLRASERPG | JERASRLPG | JAERSALRG | JRLASAPLG | JLAPSARAG | JPERSRLAG | JAARSAPAG |
| JRELSPRLG | JLRASERLG | JERASRLLG | JAERSAARG | JRLASAPAG | JLAPSALRG | JPERSRALG | JAARSALPG |
| JRELSPRAG | JLRASERAG | JERASRLAG | JAEPSRRLG | JRLASALPG | JLAPSAARG | JPERSRPAG | JAARSALLG |
| JRELSPLRG | JLRASEPRG | JERASRAPG | JAEPSRRAG | JRLASALAG | JLALSRREG | JPERSLRLG | JAARSALAG |
| JRELSPARG | JLRASELRG | JERASRALG | JAEPSRLRG | JRLASAAPG | JLALSRERG | JPERSLRAG | JAARSAAPG |
| JRELSLRPG | JLRASEARG | JERASRAAG | JAEPSRARG | JRLASAALG | JLALSRPAG | JPERSLLRG | JAARSPALG |
| JRELSLRAG | JLRASPREG | JERASPRLG | JAEPSLRRG | JRLASAAAG | JLALSRAPG | JPERSLARG | JAARSAAAG |
| JRELSLPRG | JLRASPERG | JERASPRAG | JAEPSARRG | JRARSEPLG | JLALSRAAG | JPERSARLG | JAAESRRPG |
| JRELSLARG | JLRASPLAG | JERASPLRG | JAELSRRPG | JRARSEPAG | JLALSERRG | JPERSARAG | JAAESRRLG |
| JRELSARPG | JLRASPALG | JERASPARG | JAELSRRLG | JRARSELPG | JLALSPRAG | JPERSALRG | JAAESRRAG |
| JRELSARLG | JLRASPAAG | JERASLRPG | JAELSRRAG | JRARSELLG | JLALSPARG | JPERSAARG | JAAESRPRG |
| JRELSARAG | JLRASLREG | JERASLRLG | JAELSRPRG | JRARSELAG | JLALSARPG | JPELSRRLG | JAAESRLRG |
| JRELSAPRG | JLRASLERG | JERASLRAG | JAELSRLRG | JRARSEAPG | JLALSARAG | JPELSRRAG | JAAESRARG |
| JRELSALRG | JLRASLPAG | JERASLPRG | JAELSRARG | JRARSEALG | JLALSAPRG | JPELSRLRG | JAAESPRRG |
| JRELSAARG | JLRASLAPG | JERASLLRG | JAELSPRRG | JRARSEAAG | JLALSAARG | JPELSRARG | JAAESLRRG |
| JREASRPLG | JLRASLAAG | JERASLARG | JAELSLRRG | JRARSPELG | JLPASRREG | JPELSLRRG | JAAESARRG |
| JREASRPAG | JLRASAREG | JERASARPG | JAELSARRG | JRARSPEAG | JLAASRERG | JPELSARRG | JAAPSRREG |
| JREASRLPG | JLRASAERG | JERASARLG | JAEASRRPG | JRARSPLEG | JLAASRPLG | JPEASRRLG | JAAPSRERG |
| JREASRLLG | JLRASAPLG | JERASARAG | JAEASRRLG | JRARSPAEG | JLAASRPAG | JPEASRRAG | JAAPSRLLG |
| JREASRLAG | JLRASAPAG | JERASAPRG | JAEASRRAG | JRARSLEPG | JLAASRLPG | JPEASRLRG | JAAPSRLAG |
| JREASRAPG | JLRASALPG | JERASALRG | JAEASRPRG | JRARSLELG | JLAASRLAG | JPEASRARG | JAAPSRALG |
| JREASRALG | JLRASALAG | JERASAARG | JAEASRLRG | JRARSLEAG | JLAASRAPG | JPEASLRRG | JAAPSRAAG |
| JREASRAAG | JLRASAAPG | JEPRSRLLG | JAEASRARG | JRARSLPEG | JLAASRALG | JPEASARRG | JAAPSERRG |
| JREASPRLG | JLRASAALG | JEPRSRLAG | JAEASPRRG | JRARSLLEG | JLAASRAAG | JPLRSRELG | JAAPSLRLG |
| JREASPRAG | JLRASAAAG | JEPRSRALG | JAEASLRRG | JRARSLAEG | JLAASERRG | JPLRSREAG | JAAPSLRAG |
| JREASPLRG | JLERSRPLG | JEPRSRAAG | JAEASARRG | JRARSAEPG | JLAASPRLG | JPLRSRLEG | JAAPSLLRG |
| JREASPARG | JLERSRPAG | JEPRSLRLG | JAPRSRELG | JRARSAELG | JLAASPRAG | JPLRSRAEG | JAAPSLARG |
| JREASLRPG | JLERSRLPG | JEPRSLRAG | JAPRSREAG | JRARSAEAG | JLAASPLRG | JPLRSERLG | JAAPSARLG |
| JREASLRLG | JLERSRLAG | JEPRSLLRG | JAPRSRLEG | JRARSAPEG | JLAASPARG | JPLRSERAG | JAAPSARAG |
| JREASLRAG | JLERSRAPG | JEPRSLARG | JAPRSRAEG | JRARSALEG | JLAASLRPG | JPLRSELRG | JAAPSALRG |
| JREASLPRG | JLERSRALG | JEPRSARLG | JAPRSERLG | JRARSAAEG | JLAASLRAG | JPLRSEARG | JAAPSAARG |
| JREASLLRG | JLERSRAAG | JEPRSARAG | JAPRSERAG | JRAESRPLG | JLAASLPRG | JPLRSLREG | JAALSRREG |
| JREASLARG | JLERSPRLG | JEPRSALRG | JAPRSELRG | JRAESRPAG | JLAASLARG | JPLRSLERG | JAALSRERG |
| JREASARPG | JLERSPRAG | JEPRSAARG | JAPRSEARG | JRAESRLPG | JLAASARPG | JPLRSLAAG | JAALSRPLG |
| JREASARLG | JLERSPLRG | JEPLSRRLG | JAPRSLREG | JRAESRLLG | JLAASARLG | JPLRSAREG | JAALSRPAG |
| JREASARAG | JLERSPARG | JEPLSRRAG | JAPRSLERG | JRAESRLAG | JLAASARAG | JPLRSAERG | JAALSRLPG |
| JREASAPRG | JLERSLRPG | JEPLSRLRG | JAPRSLLAG | JRAESRAPG | JLAASAPRG | JPLRSALAG | JAALSRLAG |
| JREASALRG | JLERSLRAG | JEPLSRARG | JAPRSLALG | JRAESRALG | JLAASALRG | JPLRSAALG | JAALSRAPG |
| JREASAARG | JLERSLPRG | JEPLSLRRG | JAPRSLAAG | JRAESRPAG | JLAASAARG | JPLRSAAAG | JAALSRALG |
| JRPRSELLG | JLERSLARG | JEPLSARRG | JAPRSAREG | JRAESPRLG | JARRSEPLG | JPLESRRLG | JAALSRAAG |
| JRPRSELAG | JLERSARPG | JEPASRRLG | JAPRSAERG | JRAESPRAG | JARRSEPAG | JPLESRRAG | JAALSERRG |
| JRPRSEALG | JLERSARLG | JEPASRRAG | JAPRSALLG | JRAESPLRG | JARRSELPG | JPLESRLRG | JAALSPRLG |
| JRPRSEAAG | JLERSARAG | JEPASRLRG | JAPRSALAG | JRAESPARG | JARRSELLG | JPLESRARG | JAALSPRAG |
| JRPRSLELG | JLERSAPRG | JEPASRARG | JAPRSAALG | JRAESLRPG | JARRSELAG | JPLESLRRG | JAALSPLRG |
| JRPRSLEAG | JLERSALRG | JEPASLRRG | JAPRSAAAG | JRAESLRLG | JARRSEAPG | JPLESARRG | JAALSPARG |
| JRPRSLLEG | JLERSAARG | JEPASARRG | JAPESRRLG | JRAESLRAG | JARRSEALG | JPLLSRREG | JAALSLRPG |
| JRPRSLAEG | JLEPSRRLG | JELRSRPLG | JAPESRRAG | JRAESLPRG | JARRSEAAG | JPLLSRERG | JAALSLRAG |
| JRPRSAELG | JLEPSRRAG | JELRSRPAG | JAPESRLRG | JRAESLLRG | JARRSPELG | JPLLSRAAG | JAALSLPRG |
| JRPRSAEAG | JLEPSRLRG | JELRSRLPG | JAPESRARG | JRAESLARG | JARRSPEAG | JPLLSERRG | JAALSLARG |
| JRPRSALEG | JLEPSRARG | JELRSRLAG | JAPESLRRG | JRAESARPG | JARRSPLEG | JPLLSARAG | JAALSARPG |
| JRPRSAAEG | JLEPSLRRG | JELRSRAPG | JAPESARRG | JRAESARLG | JARRSPAEG | JPLLSAARG | JAALSARLG |
| JRPESRLLG | JLEPSARRG | JELRSRALG | JAPLSRREG | JRAESARAG | JARRSLEPG | JPLASRREG | JAALSARAG |
| JRPESRLAG | JLELSRRPG | JELRSRPAG | JAPLSRERG | JRAESAPRG | JARRSLELG | JPLASRERG | JAALSAPRG |
| JRPESRALG | JLELSRRAG | JELRSPRLG | JAPLSRLAG | JRAESALRG | JARRSLEAG | JPLASRLAG | JAALSALRG |
| JRPESRAAG | JLELSRPRG | JELRSPRAG | JAPLSRALG | JRAESAARG | JARRSLPEG | JPLASRALG | JAALSAARG |
| JRPESLRLG | JLELSRARG | JELRSPLRG | JAPLSRAAG | JRAPSRELG | JARRSLLEG | JPLASRAAG | JAAASRREG |
| JRPESLRAG | JLELSPRRG | JELRSPARG | JAPLSERRG | JRAPSREAG | JARRSLAEG | JPLASERRG | JAAASRERG |
| JRPESLLRG | JLELSARRG | JELRSLRPG | JAPLSLRAG | JRAPSRLEG | JARRSAEPG | JPLASLRAG | JAAASRPLG |
| JRPESLARG | JLEASRRPG | JELRSLRAG | JAPLSLARG | JRAPSRAEG | JARRSAELG | JPLASLARG | JAAASRPAG |
| JRPESARLG | JLEASRRLG | JELRSLPRG | JAPLSARLG | JRAPSERLG | JARRSAEAG | JPLASARLG | JAAASRLPG |
| JRPESARAG | JLEASRRAG | JELRSLARG | JAPLSARAG | JRAPSERAG | JARRSAPEG | JPLASARAG | JAAASRLLG |
| JRPESALRG | JLEASRPRG | JELRSARPG | JAPLSALRG | JRAPSELRG | JARRSALEG | JPLASALRG | JAAASRLAG |
| JRPESAARG | JLEASRLRG | JELRSARLG | JAPLSAARG | JRAPSEARG | JARRSAAEG | JPLASAARG | JAAASRAPG |
| JRPLSRELG | JLEASRARG | JELRSARAG | JAPASRREG | JRAPSLREG | JARESRPLG | JPARSRELG | JAAASRALG |
| JRPLSREAG | JLEASPRRG | JELRSAPRG | JAPASRERG | JRAPSLERG | JARESRPAG | JPARSREAG | JAAASRAAG |
| JRPLSRLEG | JLEASLRRG | JELRSALRG | JAPASRLLG | JRAPSLLAG | JARESRLPG | JPARSRLEG | JAAASERRG |
| JRPLSRAEG | JLEASARRG | JELRSAARG | JAPASRLAG | JRAPSLALG | JARESRLLG | JPARSRAEG | JAAASPRLG |
| JRPLSERLG | JLPRSRELG | JELPSRRLG | JAPASRALG | JRAPSLAAG | JARESRLAG | JPARSERLG | JAAASPRAG |
| JRPLSERAG | JLPRSREAG | JELPSRRAG | JAPASRAAG | JRAPSAREG | JARESRAPG | JPARSERAG | JAAASPLRG |
| JRPLSELRG | JLPRSRLEG | JELPSRLRG | JAPASERRG | JRAPSAERG | JARESRALG | JPARSELRG | JAAASPARG |
| JRPLSEARG | JLPRSRAEG | JELPSRARG | JAPASLRLG | JRAPSALLG | JARESRAAG | JPARSEARG | JAAASLRPG |
| JRPLSLREG | JLPRSERLG | JELPSLRRG | JAPASLRAG | JRAPSALAG | JARESPRLG | JPARSLREG | JAAASLRLG |
| JRPLSLERG | JLPRSERAG | JELPSARRG | JAPASLLRG | JRAPSAALG | JARESPRAG | JPARSLERG | JAAASLRAG |
| JRPLSLAAG | JLPRSELRG | JELLSRRPG | JAPASLARG | JRAPSAAAG | JARESPLRG | JPARSLLAG | JAAASLPRG |
| JRPLSAREG | JLPRSEARG | JELLSRRAG | JAPASARLG | JRALSREPG | JARESPARG | JPARSLALG | JAAASLLRG |
| JRPLSAERG | JLPRSLREG | JELLSRPRG | JAPASARAG | JRALSRELG | JARESLRPG | JPARSLAAG | JAAASLARG |
| JRPLSALAG | JLPRSLERG | JELLSRARG | JAPASALRG | JRALSREAG | JARESLRLG | JPARSAREG | JAAASARPG |
| JRPLSAALG | JLPRSLAAG | JELLSPRRG | JAPASAARG | JRALSRPEG | JARESLRAG | JPARSAERG | JAAASARLG |
| JRPLSAAAG | JLPRSAREG | JELLSARRG | JALRSREPG | JRALSRLEG | JARESLPRG | JPARSALLG | JAAASARAG |
| JRPASRELG | JLPRSAERG | JELASRRPG | JALRSRELG | JRALSRAEG | JARESLLRG | JPARSALAG | JAAASAPRG |
| JRPASREAG | JLPRSALAG | JELASRRLG | JALRSREAG | JRALSERPG | JARESLARG | JPARSAALG | JAAASALRG |
| JRPASRLEG | JLPRSAALG | JELASRRAG | JALRSRPEG | JRALSERLG | JARESARPG | JPARSAAAG | JAAASAARG |
| JRPASRAEG | JLPRSAAAG | JELASRPRG | JALRSRLEG | JRALSERAG | JARESARLG | | |

The peptide sequences in this library are SEQ ID Nos 1-1566.

The following table (Table 6) contains an exemplary peptide library wherein the peptides in Table 5 above have been converted to the binary format for the classes B, Z and U.

TABLE 6
Exemplary Peptide Library in Partial Binary Format

| | | | | | | | |
|---|---|---|---|---|---|---|---|
| JBBZSPUUG | JPAZSBBUG | JBAUSZPBG | JABZSABAG | JBPASZBUG | JUPZSBBUG | JZUASBUBG | JAUBSBAZG |
| JBBZSPUAG | JPAZSBBAG | JBAUSZUBG | JABZSAPBG | JBPASZBAG | JUPZSBBAG | JZUASBABG | JAUBSZBPG |
| JBBZSPAUG | JPAZSBUBG | JBAUSZABG | JABZSAUBG | JBPASZUBG | JUPZSBUBG | JZUASPBBG | JAUBSZBUG |
| JBBZSPAAG | JPAZSBABG | JBAUSPBZG | JABZSAABG | JBPASZABG | JUPZSBABG | JZUASUBBG | JAUBSZBAG |
| JBBZSUPUG | JPAZSUBBG | JBAUSPZBG | JABPSBZUG | JBPASUBZG | JUPZSUBBG | JZUASABBG | JAUBSZPBG |
| JBBZSUPAG | JPAZSABBG | JBAUSPUAG | JABPSBZAG | JBPASUZBG | JUPZSABBG | JZABSBPUG | JAUBSZUBG |
| JBBZSUUPG | JPAUSBBZG | JBAUSPAUG | JABPSBUZG | JBPASUUAG | JUPUSBBZG | JZABSBPAG | JAUBSZABG |
| JBBZSUUAG | JPAUSBZBG | JBAUSPAAG | JABPSBAZG | JBPASUAUG | JUPUSBZBG | JZABSBUPG | JAUBSPBZG |
| JBBZSUAPG | JPAUSBUAG | JBAUSUBZG | JABPSZBUG | JBPASUAAG | JUPUSBAAG | JZABSBUUG | JAUBSPZBG |
| JBBZSUAUG | JPAUSBAUG | JBAUSUZBG | JABPSZBAG | JBPASABZG | JUPUSZBBG | JZABSBUAG | JAUBSPUAG |
| JBBZSUAAG | JPAUSBAAG | JBAUSUPAG | JABPSZUBG | JBPASAZBG | JUPUSABAG | JZABSBAPG | JAUBSPAUG |
| JBBZSAPUG | JPAUSZBBG | JBAUSUAPG | JABPSZABG | JBPASAUUG | JUPUSAABG | JZABSBAUG | JAUBSPAAG |
| JBBZSAPAG | JPAUSUBAG | JBAUSUAAG | JABPSUBZG | JBPASAUAG | JUPASBBZG | JZABSBAAG | JAUBSUBZG |
| JBBZSAUPG | JPAUSUABG | JBAUSABZG | JABPSUZBG | JBPASAAUG | JUPASBZBG | JZABSPBUG | JAUBSUZBG |
| JBBZSAUUG | JPAUSABUG | JBAUSAZBG | JABPSUUAG | JBPASAAAG | JUPASBUAG | JZABSPBAG | JAUBSUPAG |
| JBBZSAUAG | JPAUSABAG | JBAUSAPUG | JABPSUAUG | JBUBSZPUG | JUPASBAUG | JZABSPUBG | JAUBSUAPG |
| JBBZSAAPG | JPAUSAUBG | JBAUSAPAG | JABPSUAAG | JBUBSZPAG | JUPASBAAG | JZABSPABG | JAUBSUAAG |
| JBBZSAAUG | JPAUSAABG | JBAUSAUPG | JABPSABZG | JBUBSZUPG | JUPASZBBG | JZABSUBPG | JAUBSABZG |
| JBBZSAAAG | JPAASBBZG | JBAUSAUAG | JABPSAZBG | JBUBSZUAG | JUPASUBAG | JZABSUBUG | JAUBSAZBG |
| JBBPSZUUG | JPAASBZBG | JBAUSAAPG | JABPSAUUG | JBUBSZAPG | JUPASUABG | JZABSUBAG | JAUBSAPUG |
| JBBPSZUAG | JPAASBUUG | JBAUSAAUG | JABPSAUAG | JBUBSZAUG | JUPASABUG | JZABSUPBG | JAUBSAPAG |
| JBBPSZAUG | JPAASBUAG | JBAUSAAAG | JABPSAAUG | JBUBSZAAG | JUPASABAG | JZABSUUBG | JAUBSAUPG |
| JBBPSZAAG | JPAASBAUG | JBAASBZPG | JABPSAAAG | JBUBSPZUG | JUPASAUBG | JZABSUABG | JAUBSAUAG |
| JBBPSUZUG | JPAASBAAG | JBAASBZUG | JABUSBZPG | JBUBSPZAG | JUPASAABG | JZABSABPG | JAUBSAAPG |
| JBBPSUZAG | JPAASZBBG | JBAASBZAG | JABUSBZUG | JBUBSPUZG | JUUBSBZPG | JZABSABUG | JAUBSAAUG |
| JBBPSUUZG | JPAASUBUG | JBAASBPZG | JABUSBZAG | JBUBSPAZG | JUUBSBZAG | JZABSABAG | JAUBSAAAG |
| JBBPSUAZG | JPAASUBAG | JBAASBUZG | JABUSBPZG | JBUBSUZPG | JUUBSBPZG | JZABSAPBG | JAUZSBBPG |
| JBBPSAZUG | JPAASUUBG | JBAASBAZG | JABUSBUZG | JBUBSUZAG | JUUBSBAZG | JZABSAUBG | JAUZSBBUG |
| JBBPSAZAG | JPAASUABG | JBAASZBPG | JABUSBAZG | JBUBSUPZG | JUUBSZBPG | JZABSAABG | JAUZSBBAG |
| JBBPSAUZG | JPAASABUG | JBAASZBUG | JABUSZBPG | JBUBSUAZG | JUUBSZBAG | JZAPSBBUG | JAUZSBPBG |
| JBBPSAAZG | JPAASABAG | JBAASZBAG | JABUSZBUG | JBUBSAZPG | JUUBSZPBG | JZAPSBBAG | JAUZSBUBG |
| JBBUSZPUG | JPAASAUBG | JBAASZPBG | JABUSZBAG | JBUBSAZUG | JUUBSZABG | JZAPSBUBG | JAUZSBABG |
| JBBUSZPAG | JPAASAABG | JBAASZUBG | JABUSZPBG | JBUBSAZAG | JUUBSPBZG | JZAPSBABG | JAUZSPBBG |
| JBBUSZUPG | JUBBSZPUG | JBAASZABG | JABUSZUBG | JBUBSAPZG | JUUBSPZBG | JZAPSUBBG | JAUZSUBBG |
| JBBUSZUAG | JUBBSZPAG | JBAASPBZG | JABUSZABG | JBUBSAUZG | JUUBSPAAG | JZAPSABBG | JAUZSABBG |
| JBBUSZAPG | JUBBSZUPG | JBAASPZBG | JABUSPBZG | JBUBSAAZG | JUUBSABZG | JZAUSBBPG | JAUPSBBZG |
| JBBUSZAUG | JUBBSZUAG | JBAASPUUG | JABUSPZBG | JBUZSBPUG | JUUBSAZBG | JZAUSBBUG | JAUPSBZBG |
| JBBUSZAAG | JUBBSZAPG | JBAASPUAG | JABUSPUAG | JBUZSBPAG | JUUBSAPAG | JZAUSBBAG | JAUPSBUAG |
| JBBUSPZUG | JUBBSZAUG | JBAASPAUG | JABUSPAUG | JBUZSBUPG | JUUBSAAPG | JZAUSBPBG | JAUPSBAUG |
| JBBUSPZAG | JUBBSZAAG | JBAASPAAG | JABUSPAAG | JBUZSBUAG | JUUBSAAAG | JZAUSBUBG | JAUPSBAAG |
| JBBUSPUZG | JUBBSPZUG | JBAASUBZG | JABUSUBZG | JBUZSBAPG | JUUZSBBPG | JZAUSBABG | JAUPSZBBG |
| JBBUSPAZG | JUBBSPZAG | JBAASUZBG | JABUSUZBG | JBUZSBAUG | JUUZSBBAG | JZAUSPBBG | JAUPSUBAG |
| JBBUSUZPG | JUBBSPUZG | JBAASUPUG | JABUSUPAG | JBUZSBAAG | JUUZSBPBG | JZAUSUBBG | JAUPSUABG |
| JBBUSUZAG | JUBBSPAZG | JBAASUPAG | JABUSUAPG | JBUZSPBUG | JUUZSBABG | JZAUSABBG | JAUPSABUG |
| JBBUSUPZG | JUBBSUZPG | JBAASUUPG | JABUSUAAG | JBUZSPBAG | JUUZSPBBG | JZAASBBPG | JAUPSABAG |
| JBBUSUAZG | JUBBSUZAG | JBAASUUAG | JABUSABZG | JBUZSPUBG | JUUZSABBG | JZAASBBUG | JAUPSAUBG |
| JBBUSAZPG | JUBBSUPZG | JBAASUAPG | JABUSAZBG | JBUZSPABG | JUUPSBBZG | JZAASBBAG | JAUPSAABG |
| JBBUSAZUG | JUBBSUAZG | JBAASUAUG | JABUSAPUG | JBUZSUBPG | JUUPSBZBG | JZAASBPBG | JAUUSBBZG |
| JBBUSAZAG | JUBBSAZPG | JBAASUAAG | JABUSAPAG | JBUZSUBAG | JUUPSBAAG | JZAASBUBG | JAUUSBZBG |
| JBBUSAPZG | JUBBSAZUG | JBAASABZG | JABUSAUPG | JBUZSUPBG | JUUPSZBBG | JZAASBABG | JAUUSBPAG |
| JBBUSAUZG | JUBBSAZAG | JBAASAZBG | JABUSAUAG | JBUZSUABG | JUUPSABAG | JZAASPBBG | JAUUSBAPG |
| JBBUSAAZG | JUBBSAPZG | JBAASAPUG | JABUSAAPG | JBUZSABPG | JUUPSAABG | JZAASUBBG | JAUUSBAAG |
| JBBASZPUG | JUBBSAUZG | JBAASAPAG | JABUSAAUG | JBUZSABUG | JUUASBBZG | JZAASABBG | JAUUSZBBG |
| JBBASZPAG | JUBBSAAZG | JBAASAUPG | JABUSAAAG | JBUZSABAG | JUUASBZBG | JPBBSZUUG | JAUUSPBAG |
| JBBASZUPG | JUBZSBPUG | JBAASAUUG | JABASBZPG | JBUZSAPBG | JUUASBPAG | JPBBSZUAG | JAUUSPABG |
| JBBASZUUG | JUBZSBPAG | JBAASAUAG | JABASBZUG | JBUZSAUBG | JUUASBAPG | JPBBSZAUG | JAUUSABPG |
| JBBASZUAG | JUBZSBUPG | JBAASPAPG | JABASBZAG | JBUZSAABG | JUUASBAAG | JPBBSZAAG | JAUUSABAG |
| JBBASZAPG | JUBZSBUAG | JBAASAAUG | JABASBPZG | JBUPSBZUG | JUUASZBBG | JPBBSUZUG | JAUUSAPBG |
| JBBASZAUG | JUBZSBAPG | JBAASAAAG | JABASBUZG | JBUPSBZAG | JUUASPBAG | JPBBSUZAG | JAUUSAABG |
| JBBASZAAG | JUBZSBAUG | JZBBSPUUG | JABASBAZG | JBUPSBUZG | JUUASPABG | JPBBSUUZG | JAUASBBZG |
| JBBASPZUG | JUBZSBAAG | JZBBSPUAG | JABASZBPG | JBUPSBAZG | JUUASABPG | JPBBSUAZG | JAUASBZBG |
| JBBASPZAG | JUBZSPBUG | JZBBSPAUG | JABASZBUG | JBUPSZBUG | JUUASABAG | JPBBSAZUG | JAUASBPUG |
| JBBASPUZG | JUBZSPBAG | JZBBSPAAG | JABASZBAG | JBUPSZBAG | JUUASAPBG | JPBBSAZAG | JAUASBPAG |
| JBBASPAZG | JUBZSPUBG | JZBBSUPUG | JABASZPBG | JBUPSZUBG | JUUASAABG | JPBBSAUZG | JAUASBUPG |
| JBBASUZPG | JUBZSPABG | JZBBSUPAG | JABASZUBG | JBUPSZABG | JUABSBZPG | JPBBSAAZG | JAUASBUAG |
| JBBASUZUG | JUBZSUBPG | JZBBSUUPG | JABASZABG | JBUPSUBZG | JUABSBZUG | JPBZSBUUG | JAUASBAPG |
| JBBASUZAG | JUBZSUBAG | JZBBSUUAG | JABASPBZG | JBUPSUZBG | JUABSBZAG | JPBZSBUAG | JAUASBAUG |
| JBBASUPZG | JUBZSUPBG | JZBBSUAPG | JABASPZBG | JBUPSUAAG | JUABSBPZG | JPBZSBAUG | JAUASBAAG |
| JBBASUUZG | JUBZSUABG | JZBBSUAUG | JABASPUUG | JBUPSABZG | JUABSBUZG | JPBZSBAAG | JAUASZBBG |
| JBBASUAZG | JUBZSABPG | JZBBSUAAG | JABASPUAG | JBUPSAZBG | JUABSBAZG | JPBZSUBUG | JAUASPBUG |
| JBBASAZPG | JUBZSABUG | JZBBSAPUG | JABASPAUG | JBUPSAUAG | JUABSZBPG | JPBZSUBAG | JAUASPBAG |
| JBBASAZUG | JUBZSABAG | JZBBSAPAG | JABASPAAG | JBUPSAAUG | JUABSZBUG | JPBZSUUBG | JAUASPUBG |
| JBBASAZAG | JUBZSAPBG | JZBBSAUPG | JABASUBZG | JBUPSAAAG | JUABSZBAG | JPBZSUABG | JAUASPABG |
| JBBASAPZG | JUBZSAUBG | JZBBSAUUG | JABASUZBG | JBUUSBZPG | JUABSZPBG | JPBZSABUG | JAUASUBPG |
| JBBASAUZG | JUBZSAABG | JZBBSAUAG | JABASUPUG | JBUUSBZAG | JUABSZUBG | JPBZSABAG | JAUASUBAG |
| JBBASAAZG | JUBPSBZUG | JZBBSAAPG | JABASUPAG | JBUUSBPZG | JUABSZABG | JPBZSAUBG | JAUASUPBG |
| JBZBSPUUG | JUBPSBZAG | JZBBSAAUG | JABASUUPG | JBUUSBAZG | JUABSPBZG | JPBZSAABG | JAUASUABG |
| JBZBSPUAG | JUBPSBUZG | JZBBSAAAG | JABASUUAG | JBUUSZBPG | JUABSPZBG | JPBUSBZUG | JAUASABPG |
| JBZBSPAUG | JUBPSBAZG | JZBPSBUUG | JABASUAPG | JBUUSZBAG | JUABSPUAG | JPBUSBZAG | JAUASABUG |
| JBZBSPAAG | JUBPSZBUG | JZBPSBUAG | JABASUAUG | JBUUSZPBG | JUABSPAUG | JPBUSBUZG | JAUASABAG |
| JBZBSUPUG | JUBPSZBAG | JZBPSBAUG | JABASUAAG | JBUUSZABG | JUABSPAAG | JPBUSBAZG | JAUASAPBG |
| JBZBSUPAG | JUBPSZUBG | JZBPSBAAG | JABASABZG | JBUUSPBZG | JUABSUBZG | JPBUSZBUG | JAUASAUBG |
| JBZBSUUPG | JUBPSZABG | JZBPSUBUG | JABASAZBG | JBUUSPZBG | JUABSUZBG | JPBUSZBAG | JAUASAABG |
| JBZBSUUAG | JUBPSUBZG | JZBPSUBAG | JABASAPUG | JBUUSPAAG | JUABSUPAG | JPBUSZUBG | JAABSBZPG |
| JBZBSUAPG | JUBPSUZBG | JZBPSUUBG | JABASAPAG | JBUUSABZG | JUABSUAPG | JPBUSZABG | JAABSBZUG |
| JBZBSUAUG | JUBPSUAAG | JZBPSUABG | JABASAUPG | JBUUSAZBG | JUABSUAAG | JPBUSUBZG | JAABSBZAG |
| JBZBSUAAG | JUBPSABZG | JZBPSABUG | JABASAUUG | JBUUSAPAG | JUABSABZG | JPBUSUZBG | JAABSBPZG |
| JBZBSAPUG | JUBPSAZBG | JZBPSABAG | JABASAUAG | JBUUSAAPG | JUABSAZBG | JPBUSUAAG | JAABSBUZG |
| JBZBSAPAG | JUBPSAUAG | JZBPSAUBG | JABASAAPG | JBUUSAAAG | JUABSAPUG | JPBUSABZG | JAABSBAZG |
| JBZBSAUPG | JUBPSAAUG | JZBPSAABG | JABASAAUG | JBUASBZPG | JUABSAPAG | JPBUSAZBG | JAABSZBPG |
| JBZBSAUUG | JUBPSAAAG | JZBUSBPUG | JABASPAAG | JBUASBZUG | JUABSAUPG | JPBUSAUAG | JAABSZBUG |
| JBZBSAUAG | JUBUSBZPG | JZBUSBPAG | JAZBSBPUG | JBUASBZAG | JUABSAUAG | JPBUSAAUG | JAABSZBAG |
| JBZBSAAPG | JUBUSBZAG | JZBUSBUPG | JAZBSBPAG | JBUASBPZG | JUABSAAPG | JPBUSAAAG | JAABSZPBG |
| JBZBSAAUG | JUBUSBPZG | JZBUSBUAG | JAZBSBUPG | JBUASBUZG | JUABSAAUG | JPBASBZUG | JAABSZUBG |
| JBZBSAAAG | JUBUSBAZG | JZBUSBAPG | JAZBSBUUG | JBUASBAZG | JUABSAAAG | JPBASBZAG | JAABSZABG |
| JBZPSBUUG | JUBUSZBPG | JZBUSBAUG | JAZBSBUAG | JBUASZBPG | JUAZSBBPG | JPBASBUZG | JAABSPBZG |
| JBZPSBUAG | JUBUSZBAG | JZBUSBAAG | JAZBSBAPG | JBUASZBUG | JUAZSBBUG | JPBASBAZG | JAABSPZBG |
| JBZPSBAUG | JUBUSZPBG | JZBUSPBUG | JAZBSBAUG | JBUASZBAG | JUAZSBBAG | JPBASZBUG | JAABSPUUG |
| JBZPSBAAG | JUBUSZABG | JZBUSPBAG | JAZBSBAAG | JBUASZPBG | JUAZSBPBG | JPBASZBAG | JAABSPUAG |
| JBZPSUBUG | JUBUSPBZG | JZBUSPUBG | JAZBSPBUG | JBUASZUBG | JUAZSBUBG | JPBASZUBG | JAABSPAUG |
| JBZPSUBAG | JUBUSPZBG | JZBUSPABG | JAZBSPBAG | JBUASZABG | JUAZSBABG | JPBASZABG | JAABSPAAG |
| JBZPSUUBG | JUBUSPAAG | JZBUSUBPG | JAZBSPUBG | JBUASPBZG | JUAZSPBBG | JPBASUBZG | JAABSUBZG |
| JBZPSUABG | JUBUSABZG | JZBUSUBAG | JAZBSPABG | JBUASPZBG | JUAZSUBBG | JPBASUZBG | JAABSUZBG |
| JBZPSABUG | JUBUSAZBG | JZBUSUPBG | JAZBSUBPG | JBUASPUAG | JUAZSABBG | JPBASUUAG | JAABSUPUG |
| JBZPSABAG | JUBUSAPAG | JZBUSUABG | JAZBSUBUG | JBUASPAUG | JUAPSBBZG | JPBASUAUG | JAABSUPAG |
| JBZPSAUBG | JUBUSAAPG | JZBUSABPG | JAZBSUBAG | JBUASPAAG | JUAPSBZBG | JPBASUAAG | JAABSUUPG |
| JBZPSAABG | JUBUSAAAG | JZBUSABUG | JAZBSUPBG | JBUASUBZG | JUAPSBUAG | JPBASABZG | JAABSUUAG |
| JBZUSBPUG | JUBASBZPG | JZBUSABAG | JAZBSUUBG | JBUASUZBG | JUAPSBAUG | JPBASAZBG | JAABSUAPG |
| JBZUSBPAG | JUBASBZUG | JZBUSAPBG | JAZBSUABG | JBUASUPAG | JUAPSBAAG | JPBASAUUG | JAABSUAUG |
| JBZUSBUPG | JUBASBZAG | JZBUSAUBG | JAZBSABPG | JBUASUAPG | JUAPSZBBG | JPBASAUAG | JAABSUAAG |
| JBZUSBUAG | JUBASBPZG | JZBUSAABG | JAZBSABUG | JBUASUAAG | JUAPSUBAG | JPBASAAUG | JAABSABZG |
| JBZUSBAPG | JUBASBUZG | JZBASBPUG | JAZBSABAG | JBUASABZG | JUAPSUABG | JPBASAAAG | JAABSAZBG |
| JBZUSBAUG | JUBASBAZG | JZBASBPAG | JAZBSAPBG | JBUASAZBG | JUAPSABUG | JPZBSBUUG | JAABSAPUG |
| JBZUSBAAG | JUBASZBPG | JZBASBUPG | JAZBSAUBG | JBUASAPUG | JUAPSABAG | JPZBSBUAG | JAABSAPAG |
| JBZUSPBUG | JUBASZBUG | JZBASBUUG | JAZBSAABG | JBUASAPAG | JUAPSAUBG | JPZBSBAUG | JAABSAUPG |
| JBZUSPBAG | JUBASZBAG | JZBASBUAG | JAZPSBBUG | JBUASAUPG | JUAPSAABG | JPZBSBAAG | JAABSAUUG |
| JBZUSPUBG | JUBASZPBG | JZBASBAPG | JAZPSBBAG | JBUASAUAG | JUAUSBBZG | JPZBSUBUG | JAABSAUAG |
| JBZUSPABG | JUBASZUBG | JZBASBAUG | JAZPSBUBG | JBUASAAPG | JUAUSBZBG | JPZBSUBAG | JAABSAAPG |
| JBZUSUBPG | JUBASZABG | JZBASBAAG | JAZPSBABG | JBUASAAUG | JUAUSBPAG | JPZBSUUBG | JAABSAAUG |
| JBZUSUBAG | JUBASPBZG | JZBASPBUG | JAZPSUBBG | JBUASAAAG | JUAUSBAPG | JPZBSUABG | JAABSAAAG |
| JBZUSUPBG | JUBASPZBG | JZBASPBAG | JAZPSABBG | JBABSZPUG | JUAUSBAAG | JPZBSABUG | JAAZSBBPG |
| JBZUSUABG | JUBASPUAG | JZBASPUBG | JAZUSBBPG | JBABSZPAG | JUAUSZBBG | JPZBSABAG | JAAZSBBUG |
| JBZUSABPG | JUBASPAUG | JZBASPABG | JAZUSBBUG | JBABSZUPG | JUAUSPBAG | JPZBSAUBG | JAAZSBBAG |
| JBZUSABUG | JUBASPAAG | JZBASUBPG | JAZUSBBAG | JBABSZUUG | JUAUSPABG | JPZBSAABG | JAAZSBPBG |
| JBZUSABAG | JUBASUBZG | JZBASUBUG | JAZUSBPBG | JBABSZUAG | JUAUSABPG | JPZUSBBUG | JAAZSBUBG |
| JBZUSAPBG | JUBASUZBG | JZBASUBAG | JAZUSBUBG | JBABSZAPG | JUAUSABAG | JPZUSBBAG | JAAZSBABG |
| JBZUSAUBG | JUBASUPAG | JZBASUPBG | JAZUSBABG | JBABSZAUG | JUAUSAPBG | JPZUSBUBG | JAAZSPBBG |
| JBZUSAABG | JUBASUAPG | JZBASUUBG | JAZUSPBBG | JBABSZAAG | JUAUSAABG | JPZUSBABG | JAAZSUBBG |
| JBZASBPUG | JUBASUAAG | JZBASUABG | JAZUSUBBG | JBABSPZUG | JUAASBBZG | JPZUSUBBG | JAAZSABBG |
| JBZASBPAG | JUBASABZG | JZBASABPG | JAZUSABBG | JBABSPZAG | JUAASBZBG | JPZUSABBG | JAAPSBBZG |
| JBZASBUPG | JUBASAZBG | JZBASABUG | JAZASBBPG | JBABSPUZG | JUAASBPUG | JPZASBBUG | JAAPSBZBG |
| JBZASBUUG | JUBASAPUG | JZBASABAG | JAZASBBUG | JBABSPAZG | JUAASBPAG | JPZASBBAG | JAAPSBUUG |
| JBZASBUAG | JUBASAPAG | JZBASAPBG | JAZASBBAG | JBABSUZPG | JUAASBUPG | JPZASBUBG | JAAPSBUAG |
| JBZASBAPG | JUBASAUPG | JZBASAUBG | JAZASBPBG | JBABSUZUG | JUAASBUAG | JPZASBABG | JAAPSBAUG |
| JBZASBAUG | JUBASAUAG | JZBASAABG | JAZASBUBG | JBABSUZAG | JUAASBAPG | JPZASUBBG | JAAPSBAAG |
| JBZASBAAG | JUBASAAPG | JZPBSBUUG | JAZASBABG | JBABSUPZG | JUAASBAUG | JPZASABBG | JAAPSZBBG |
| JBZASPBUG | JUBASAAUG | JZPBSBUAG | JAZASPBBG | JBABSUUZG | JUAASBAAG | JPUBSBZUG | JAAPSUBUG |
| JBZASPBAG | JUBASAAAG | JZPBSBAUG | JAZASUBBG | JBABSUAZG | JUAASZBBG | JPUBSBZAG | JAAPSUBAG |
| JBZASPUBG | JUZBSBPUG | JZPBSBAAG | JAZASABBG | JBABSAZPG | JUAASPBUG | JPUBSBUZG | JAAPSUUBG |
| JBZASPABG | JUZBSBPAG | JZPBSUBUG | JAPBSBZUG | JBABSAZUG | JUAASPBAG | JPUBSBAZG | JAAPSUABG |
| JBZASUBPG | JUZBSBUPG | JZPBSUBAG | JAPBSBZAG | JBABSAZAG | JUAASPUBG | JPUBSZBUG | JAAPSABUG |
| JBZASUBUG | JUZBSBUAG | JZPBSUUBG | JAPBSBUZG | JBABSAPZG | JUAASPABG | JPUBSZBAG | JAAPSABAG |
| JBZASUBAG | JUZBSBAPG | JZPBSUABG | JAPBSBAZG | JBABSAUZG | JUAASUBPG | JPUBSZUBG | JAAPSAUBG |
| JBZASUPBG | JUZBSBAUG | JZPBSABUG | JAPBSZBUG | JBABSAAZG | JUAASUBAG | JPUBSZABG | JAAPSAABG |
| JBZASUUBG | JUZBSBAAG | JZPBSABAG | JAPBSZBAG | JBAZSBPUG | JUAASUPBG | JPUBSUBZG | JAAUSBBZG |
| JBZASUABG | JUZBSPBUG | JZPBSAUBG | JAPBSZUBG | JBAZSBPAG | JUAASUABG | JPUBSUZBG | JAAUSBZBG |
| JBZASABPG | JUZBSPBAG | JZPBSPABG | JAPBSZABG | JBAZSBUPG | JUAASABPG | JPUBSUAAG | JAAUSBPUG |
| JBZASABUG | JUZBSPUBG | JZPUSBBUG | JAPBSUBZG | JBAZSBUUG | JUAASABUG | JPUBSABZG | JAAUSBPAG |
| JBZASABAG | JUZBSPABG | JZPUSBBAG | JAPBSUZBG | JBAZSBUAG | JUAASABAG | JPUBSAZBG | JAAUSBUPG |
| JBZASAPBG | JUZBSUBPG | JZPUSBUBG | JAPBSUUAG | JBAZSBAPG | JUAASAPBG | JPUBSAUAG | JAAUSBUAG |
| JBZASAUBG | JUZBSUBAG | JZPUSBABG | JAPBSUAUG | JBAZSBAUG | JUAASAUBG | JPUBSAAUG | JAAUSBAPG |
| JBZASAABG | JUZBSUPBG | JZPUSUBBG | JAPBSUAAG | JBAZSBAAG | JUAASAABG | JPUBSAAAG | JAAUSBAUG |
| JBPBSZUUG | JUZBSUABG | JZPUSABBG | JAPBSABZG | JBAZSPBUG | JABBSZPUG | JPUZSBBUG | JAAUSBAAG |
| JBPBSZUAG | JUZBSABPG | JZPASBBUG | JAPBSAZBG | JBAZSPBAG | JABBSZPAG | JPUZSBBAG | JAAUSZBBG |
| JBPBSZAUG | JUZBSABUG | JZPASBBAG | JAPBSAUUG | JBAZSPUBG | JABBSZUPG | JPUZSBUBG | JAAUSPBUG |
| JBPBSZAAG | JUZBSABAG | JZPASBUBG | JAPBSAUAG | JBAZSPABG | JABBSZUUG | JPUZSBABG | JAAUSPBAG |
| JBPBSUZUG | JUZBSAPBG | JZPASBABG | JAPBSAAUG | JBAZSUBPG | JABBSZUAG | JPUZSUBBG | JAAUSPUBG |
| JBPBSUZAG | JUZBSAUBG | JZPASUBBG | JAPBSAAAG | JBAZSUBUG | JABBSZAPG | JPUZSABBG | JAAUSPABG |
| JBPBSUUZG | JUZBSAABG | JZPASABBG | JAPZSBBUG | JBAZSUBAG | JABBSZAUG | JPUUSBBZG | JAAUSUBPG |
| JBPBSUAZG | JUZPSBBUG | JZUBSBPUG | JAPZSBBAG | JBAZSUPBG | JABBSZAAG | JPUUSBZBG | JAAUSUBAG |
| JBPBSAZUG | JUZPSBBAG | JZUBSBPAG | JAPZSBUBG | JBAZSUUBG | JABBSPZUG | JPUUSBAAG | JAAUSUPBG |
| JBPBSAZAG | JUZPSBUBG | JZUBSBUPG | JAPZSBABG | JBAZSUABG | JABBSPZAG | JPUUSZBBG | JAAUSUABG |
| JBPBSAUZG | JUZPSBABG | JZUBSBUAG | JAPZSUBBG | JBAZSABPG | JABBSPUZG | JPUUSABAG | JAAUSABPG |
| JBPBSAAZG | JUZPSUBBG | JZUBSBAPG | JAPZSABBG | JBAZSABUG | JABBSPAZG | JPUUSAABG | JAAUSABUG |
| JBPZSBUUG | JUZPSABBG | JZUBSBAUG | JAPUSBBZG | JBAZSABAG | JABBSUZPG | JPUASBBZG | JAAUSABAG |
| JBPZSBUAG | JUZUSBBPG | JZUBSBAAG | JAPUSBZBG | JBAZSAPBG | JABBSUZUG | JPUASBZBG | JAAUSAPBG |
| JBPZSBAUG | JUZUSBBAG | JZUBSPBUG | JAPUSBUAG | JBAZSAUBG | JABBSUZAG | JPUASBUAG | JAAUSAUBG |
| JBPZSBAAG | JUZUSBPBG | JZUBSPBAG | JAPUSBAUG | JBAZSAABG | JABBSUPZG | JPUASBAUG | JAAUSAABG |
| JBPZSUBUG | JUZUSBABG | JZUBSPUBG | JAPUSBPAG | JBAPSBZUG | JABBSUUZG | JPUASBAAG | JAAASBBZG |
| JBPZSUBAG | JUZUSPBBG | JZUBSPABG | JAPUSZBBG | JBAPSBZAG | JABBSUAZG | JPUASZBBG | JAAASBZBG |
| JBPZSUUBG | JUZUSABBG | JZUBSUBPG | JAPUSUBAG | JBAPSBUZG | JABBSAZPG | JPUASUBAG | JAAASBPUG |
| JBPZSUABG | JUZASBBPG | JZUBSUBAG | JAPUSUABG | JBAPSBAZG | JABBSAZUG | JPUASUABG | JAAASBPAG |
| JBPZSABUG | JUZASBBUG | JZUBSUPBG | JAPUSABUG | JBAPSZBUG | JABBSAZAG | JPUASABUG | JAAASBUPG |
| JBPZSABAG | JUZASBBAG | JZUBSUABG | JAPUSABAG | JBAPSZBAG | JABBSAPZG | JPUASABAG | JAAASBUUG |
| JBPZSAUBG | JUZASBPBG | JZUBSABPG | JAPUSAUBG | JBAPSZUBG | JABBSAUZG | JPUASAUBG | JAAASBUAG |
| JBPZSAABG | JUZASBUBG | JZUBSABUG | JAPUSAABG | JBAPSZABG | JABBSAAZG | JPUASAABG | JAAASBAPG |
| JBPUSBZUG | JUZASBABG | JZUBSABAG | JAPASBBZG | JBAPSUBZG | JABZSBPUG | JPABSBZUG | JAAASBAUG |
| JBPUSBZAG | JUZASPBBG | JZUBSAPBG | JAPASBZBG | JBAPSUZBG | JABZSBPAG | JPABSBZAG | JAAASBAAG |
| JBPUSBUZG | JUZASUBBG | JZUBSAUBG | JAPASBUUG | JBAPSUUAG | JABZSBUPG | JPABSBUZG | JAAASZBBG |
| JBPUSBAZG | JUZASABBG | JZUBSAABG | JAPASBUAG | JBAPSUAUG | JABZSBUUG | JPABSBAZG | JAAASPBUG |
| JBPUSZBUG | JUPBSBZUG | JZUPSBBUG | JAPASBAUG | JBAPSUAAG | JABZSBUAG | JPABSZBUG | JAAASPBAG |
| JBPUSZBAG | JUPBSBZAG | JZUPSBBAG | JAPASBAAG | JBAPSABZG | JABZSBAPG | JPABSZBAG | JAAASPUBG |
| JBPUSZUBG | JUPBSBUZG | JZUPSBUBG | JAPASZBBG | JBAPSAZBG | JABZSBAUG | JPABSZUBG | JAAASPABG |
| JBPUSZABG | JUPBSBAZG | JZUPSBABG | JAPASUBUG | JBAPSAUUG | JABZSBAAG | JPABSZABG | JAAASUBPG |
| JBPUSUBZG | JUPBSZBUG | JZUPSUBBG | JAPASUBAG | JBAPSAUAG | JABZSPBUG | JPABSUBZG | JAAASUBUG |
| JBPUSUZBG | JUPBSZBAG | JZUPSABBG | JAPASUUBG | JBAPSAAUG | JABZSPBAG | JPABSUZBG | JAAASUBAG |
| JBPUSUAAG | JUPBSZUBG | JZUUSBBPG | JAPASUABG | JBAPSAAAG | JABZSPUBG | JPABSUUAG | JAAASUPBG |
| JBPUSABZG | JUPBSZABG | JZUUSBBAG | JAPASABUG | JBAUSBZPG | JABZSPABG | JPABSUAUG | JAAASUUBG |
| JBPUSAZBG | JUPBSUBZG | JZUUSBPBG | JAPASABAG | JBAUSBZUG | JABZSUBPG | JPABSUAAG | JAAASUABG |
| JBPUSAUAG | JUPBSUZBG | JZUUSBABG | JAPASAUBG | JBAUSBZAG | JABZSUBUG | JPABSABZG | JAAASABPG |
| JBPUSAAUG | JUPBSUAAG | JZUUSPBBG | JAPASAABG | JBAUSBPZG | JABZSUBAG | JPABSAZBG | JAAASABUG |
| JBPUSAAAG | JUPBSABZG | JZUUSABBG | JAUBSBZPG | JBAUSBUZG | JABZSUPBG | JPABSAUUG | JAAASABAG |
| JBPASBZUG | JUPBSAZBG | JZUASBBPG | JAUBSBZUG | JBAUSBAZG | JABZSUUBG | JPABSAUAG | JAAASAPBG |
| JBPASBUZG | JUPBSAAUG | JZUASBBAG | JAUBSBPZG | JBAUSZBUG | JABZSABPG | JPABSAAAG | JAAASAABG |
| JBPASBZAG | JUPBSAUAG | JZUASBBUG | JAUBSBZAG | JBAUSZBPG | JABZSUABG | JPABSAAUG | JAAASAUBG |
| JBPASBAZG | JUPBSAAAG | JZUASBPBG | JAUBSBUZG | JBAUSZBAG | JABZSABUG | | |

The peptide sequences in this library are SEQ ID Nos 1570-3135.

### Example 2

### Peptide Library PKA Substrate Screen Protocol Peptide Library Design

The library that was used in the PKA screen was a “binary motif library” of the following form: Lissamine-[Deg]2-X-X-X-X-X-S-X-G, where X is one of the following residues: A=alanine, P=proline, S=serine or a mixture of two residues in equal proportion and similar charge:

+1 charge residue: B=K (lysine)+R (arginine)

−1 charge residue: Z=D (aspartic acid)+E (glutamic acid)

neutral hydrophobic: U=L (leucine)+V (valine)

In the final library, there were up to 32 peptides/well. There were also limitations on the composition such as only one P allowed and net (+1) peptide charge required. In addition, the number of hydrophobic residues permitted in each peptide was no greater than 2 for solubility purposes (U≦2). The total library consisted of 1128 wells.

Assay Methods

The assay developed for the PKA substrate screen was performed as follows. The enzyme in the reaction buffer (0.017 U/μl Biomol PKAα, 10 MgCl₂, 100 mM HEPES pH 7.4, 1 mM DTT, 0.015% Brij-35, 0.25% BSA) including ATP (200 μM) was placed in a 384-well standard reaction plate. A control plate was run where no enzyme was added to the plate, only the reaction buffer. The enzymatic reaction or control (no reaction) solutions were initiated by addition of Lissamine-peptides from the peptide library at a final concentration of 20-80 μM lissamine-peptide per well. The enzymatic reaction or controls were incubated at room temperature for 35 minutes and quenched with EDTA (50 mM, pH 8). 5 μl of the reaction or control mixture was transferred to the 384-well ElectroCapture™ plate (described in Application No. PCT/US01/43508, which is herein incorporated by reference) containing 35 μL of 100 mM Tris-Borate pH8 buffer. The ElectroCapture™ plate was manually placed into the ElectroCapture™ HTS workstation lower electrode reservoir and a voltage of 160V was applied across the plate for 7 minutes. The ElectroCapture™ plate was removed from the ElectroCapture™ HTS workstation and the reaction mixture was removed/washed from the well with water via a 384-well plate Tecan™ Washer. The fluorescence was read from the top of the plate with a Tecan™ Ultra plate reader at 550 nm excitation and 612 nm emission. The fluorescence is directly proportional to the amount of phosphorylated peptide captured on the bottom of the plate.

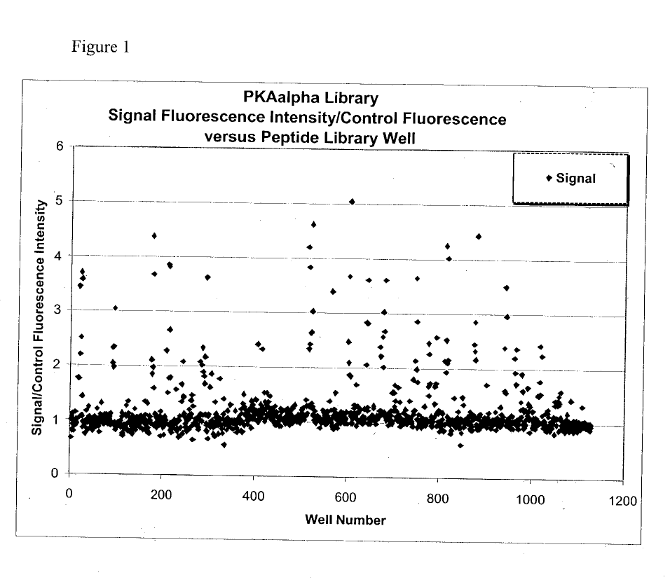

There were eleven 96-well plates in the peptide library which translated into three 384-well ElectroCapture™ plates. In addition, there were three control ElectroCapture™ plated run. To analyze the data, the ratio of the fluorescence intensity of the reaction well was divided by the fluorescence intensity of the control well to account for differences in fluorophore-labeled peptide concentration. If a signal/control ratio greater than ˜3 was observed, a phosphorylation event occurred and the well was determined a “hit”. PKA is known to phosphorylate many sequences and sequence motifs, so many wells were observed to be hits in the screen analysis (see FIG. 1). The sequences of the motifs from the well hits are shown in Table 7.

TABLE 7

Results from hits in peptide library substrate
screen with PKA

| Peptide | | Known Substrate with |
|---|---|---|
| Motif | Signal | Peptide Motif |
| BUPABSAG | 5.0 | |
| BPBBZSUG | 4.6 | |
| UBBBZSUG | 4.4 | |
| APBBASUG | 4.4 | |
| UABBASUG | 4.3 | |
| BPBAASUG | 4.2 | Crosstide: GRPRTSSFAEG |
| UABBPSUG | 4.0 | |
| AUBBASUG | 3.9 | Kemptide: LRRASLG |
| BPBAUSUG | 3.8 | |
| AUBBPSUG | 3.8 | |
| AABBPSUG | 3.7 | |
| APBBUSUG | 3.7 | |
| BUBZPSBG | 3.7 | |
| PBUABSAG | 3.7 | |
| BABBZSUG | 3.6 | |
| BZUBPSBG | 3.6 | |
| BZABUSBG | 3.6 | |
| AABBUSUG | 3.6 | |
| UPBBASUG | 3.5 | |
| AABBASUG | 3.5 | |
| BUAPBSAG | 3.4 | |
| ABBBZSUG | 3.0 | |
| BPBUASUG | 3.0 | |
| BZUBBSAG | 3.0 | |

The peptide sequences in the left column are SEQ ID Nos. 3136-3159; the crosstide sequence is SEQ ID No. 3160; and the Kemptide sequence is SEQ ID No. 3161.
Crosstide Reference: Cross et al., Nature 263: 378, 1995
Kemptide Reference: Kemp et al., J. Biol. Chem. 252: 4888, 1977

Confirmation of phosphorylated peptides present on peptide binding membranes was performed by removing the membrane off of the ElectroCapture™ plate wells by cutting the membrane with a sharp knife. The membrane was then soaked in acetonitrile to get the bound peptides into solution. Maldi-TOF mass spectrometry was performed on two wells corresponding to the Crosstide (SEQ ID No. 3141) and Kemptide (SEQ ID No. 3143) motifs and the results are shown in Table 8. The MS showed several phospho-peptides with the correct PKA recognition motif.

TABLE 8

Peptides identified by MS of peptides isolated
from ElectroCapture ™ plate membrane

| | |
|---|---|
| Peptide Sequence | Peptide MW (g/mol) |

| | |
|---|---|
| BPBAASUG Motif (Crosstide) | |
| ALRRASLG | 1753.6 |
| AVRRASLG | 1739.5 |
| AVRRASVG | 1725.7 |
| AUBBASUG Motif (Kemptide) | |
| RPRAASLG | 1737.0 |
| RPRAASVG | 1723.3 |

The peptide sequences in the left column are SEQ ID Nos 3162-3166.

Although the present invention has been described in detail with reference to the examples above, it is understood that various modifications can be made without departing from the spirit of the invention. Accordingly, the invention is limited only by the following claims. All cited patents, patent applications, and publications referred to in this application are herein incorporated by reference in their entirety.

What is claimed:

1. A peptide library comprising a plurality of different peptide molecules, wherein each peptide molecule in said library has the same net charge or a neutral charge.

2. The peptide library of claim 1, wherein said peptide molecules each have a net charge of +1 or a neutral charge.

3. The peptide library of claim 2, wherein said peptide molecules each have a net charge of +1.

4. The peptide library of claim 1, wherein said peptide molecules each have a net charge of −1 or a neutral charge.

5. The peptide library of claim 4, wherein said peptide molecules each have a net charge of −1.

6. The peptide library of claim 1, wherein said peptide molecules each have a net neutral charge.

7. The peptide library of claim 1, wherein said library comprises at least about fifty peptide molecules.

8. The peptide library of claim 1, wherein said library comprises at least about one hundred peptide molecules.

9. The peptide library of claim 1, wherein said library comprises at least about five hundred peptide molecules.

10. The peptide library of claim 1, wherein said library comprises at least about one thousand peptide molecules.

11. The peptide library of claim 1, wherein said library comprises at least about fifteen hundred peptide molecules.

12. The peptide library of claim 1, wherein said library comprises peptide molecules having at least one variable position in relation to each other.

13. The peptide library of claim 12, wherein said at least one variable position is filled by an amino acid selected from a subset of amino acids.

14. The peptide library of claim 13, wherein said subset of amino acids contains one or more amino acids selected from the group consisting of basic or positively charged amino acids, acidic or negatively charged amino acids, hydrophobic amino acids, spacing or neutral amino acids, phosphoryl-accepting amino acids, phosphoryl-donating amino acids, phosphorylated amino acids and bending amino acids.

15. The peptide library of claim 14, wherein said subset of amino acids comprises at least one basic or positively charged amino acid.

16. The peptide library of claim 15, wherein said at least one basic or positively charged amino acid is selected from the group consisting of lysine, arginine and histidine.

17. The peptide library of claim 14, wherein said subset of amino acids comprises at least one acidic or negatively charged amino acid.

18. The peptide library of claim 17, wherein said acidic or negatively charged amino acids are selected from the group consisting of aspartic acid and glutamic acid.

19. The peptide library of claim 14, wherein said subset of amino acids comprises at least one hydrophobic amino acid.

20. The peptide library of claim 19, wherein said hydrophobic amino acid is selected from the group consisting of isoleucine, leucine, methionine, phenylalanine, tryptophan, tyrosine and valine.

21. The peptide library of claim 14, wherein said subset of amino acids comprises at least one spacing or neutral amino acid.

22. The peptide library of claim 21, wherein said spacing or neutral amino acid is selected from the group consisting of glycine, alanine and homoalanine.

23. The peptide library of claim 14, wherein said subset of amino acids comprises at least one phosphoryl-accepting amino acid.

24. The peptide library of claim 23, wherein said phosphoryl-accepting amino acid is selected from the group consisting of serine, threonine, tyrosine, histidine, aspartate and lysine.

25. The peptide library of claim 14, wherein said subset of amino acids comprises at least one bending amino acid.

26. The peptide library of claim 25, wherein said bending amino acid is proline.

27. The peptide library of claim 14, wherein said subset of amino acids comprises at least one positively charged amino acid, at least one negatively charged amino acid, at least one neutral amino acid, at least one hydrophobic amino acid and at least one bending amino acid.

28. The peptide library of claim 27, wherein said subset of amino acids comprises arginine, glutamic acid, alanine, leucine and proline.

29. The peptide library of claim 13, wherein said subset of amino acids comprises at least one binary grouping of amino acids.

30. The peptide library of claim 29, wherein said at least one binary grouping is selected from the group consisting of basic or positively charged amino acids, acidic or negatively charged amino acids, hydrophobic amino acids, spacing or neutral amino acids, phosphoryl-accepting amino acids, and bending amino acids.

31. The peptide library of claim 30, wherein said binary grouping comprises two basic or positively charged amino acids.

32. The peptide library of claim 31, wherein said two basic or positively charged amino acids are lysine and arginine.

33. The peptide library of claim 30, wherein said binary grouping comprises two acidic or negatively charged amino acids.

34. The peptide library of claim 33, wherein said acidic or negatively charged amino acids are aspartic acid and glutamic acid.

35. The peptide library of claim 30, wherein said binary grouping comprises two hydrophobic amino acids.

36. The peptide library of claim 35, wherein said hydrophobic amino acids are leucine and valine.

37. The peptide library of claim 30, wherein said binary grouping comprises two spacing or neutral amino acids.

38. The peptide library of claim 37, wherein said spacing or neutral amino acids are glycine and alanine.

39. The peptide library of claim 30, wherein said binary grouping comprises two phosphoryl accepting amino acids.

40. The peptide library of claim 39, wherein said phosphoryl-accepting amino acids are serine and threonine.

41. The peptide library of claim 1, wherein the number of amino acids in each peptide molecule is no less than three.

42. The peptide library of claim 1, wherein the number of amino acids in each peptide molecule is no greater than twenty-five.

43. The peptide library of claim 1, wherein said library comprises peptide molecules having at least one non-variable position in relation to each other.

44. The peptide library of claim 43, wherein said at least one non-variable position is an ion-accepting or an ion-donating amino acid.

45. The peptide library of claim 43, wherein said at least one non-variable position is a phosphoryl accepting or phosphoryl donating amino acid.

46. The peptide library of claim 45, wherein said phosphoryl accepting or phosphoryl donating amino acid is selected from the group consisting of serine, threonine, tyrosine, histidine, aspartate and lysine.

47. The peptide library of claim 43, wherein said library comprises peptide molecules having one non-variable position wherein the number of variable amino acids amino terminal to said non-variable amino acid is from 1-5 inclusive.

48. The peptide library of claim 43, wherein said library comprises peptide molecules having one non-variable position wherein the number of variable amino acids carboxy terminal to said non-variable amino acid is from 1-5 inclusive.

49. The peptide library of claim 1, wherein said library comprises peptide molecules having one non-variable amino acid in a variable or floating position.

50. The peptide library of claim 49, wherein said at least one non-variable position is an ion-accepting or an ion-donating amino acid.

51. The peptide library of claim 50, wherein said ion-accepting or ion-donating amino acid is a phosphoryl accepting or phosphoryl donating amino acid.

52. The peptide library of claim 51, wherein said phosphoryl accepting or phosphoryl donating amino acid is selected from the group consisting of serine, threonine, tyrosine, histidine, aspartate and lysine.

53. The peptide library of claim 1, wherein the peptide molecules in said library are each associated with a detectable label.

54. The peptide library of claim 53, wherein the detectable label is a fluorophore.

55. The peptide library of claim 54, wherein the fluorophore is selected from the group consisting of Bodipy, Texas Red, DAPI, Cy-Dyes, Lissamine, fluorescein, rhodamine, phycoerythrin, free or chelated lanthanide series salts and coumarin.

56. The peptide library of claim 53, wherein the detectable label is separated from amino acids of said peptide molecules by a linker.

57. The peptide library of claim 1, wherein the individual peptide molecules comprise a linker.

58. The peptide library of claim 57, wherein said linker is selected from the group consisting of polyethylene glycol (PEG) and polysaccharides, and has a molecular weight of about 80 to 4000 Daltons.

59. The peptide library of claim 1, wherein the peptides are contained in a collection in solution.

60. The peptide library of claim 1, wherein individual peptide molecules, or mixtures of peptide molecules having at least one variable position, are segregated.

61. The peptide library of claim 60, wherein the individual peptide molecules or variable mixtures thereof are segregated into individual wells of one or more microtiter plates.

62. The peptide library of claim 61, wherein said microtiter plates fit into an electrophoretic apparatus.

63. The peptide library of claim 62, wherein said microtiter plate comprises:
(a) a plurality of substantially tubular sample wells arrayed in the sample plate; and
(b) at least one capture matrix, wherein the capture matrix is disposed in each of the sample wells proximate an end of the sample wells, and wherein the capture matrix comprises a diffusion-inhibiting material; and
wherein at least one sample well may be placed in electrical contact with at least one first electrode at the bottom end of the sample well, and with at least one second electrode at the top end of the sample well, wherein both electrodes are coupled to a power source.

64. The peptide library of claim 62, wherein said microtiter plate comprises:
at least one microstructure, each microstructure comprising a series of microstructure sections and channels, wherein each microstructure section is directly interconnected to at least one other microstructure section by at least one channel, the series comprising:
at least one sample accepting microstructure section, wherein the sample accepting section is fluidly connected to the exterior of the microstructure plate;
at least one first electrode microstructure section;
at least one second electrode microstructure section;
at least one capture microstructure section containing a capture matrix,
wherein the capture microstructure section is between the first and second electrode microstructure sections in the series; and
wherein the microstructures in the microstructure plate are formed by at least two layers of material, wherein at least one layer is a sealing plate layer which seals at least one channel or microstructure section in the assembled microstructure plate.

65. The peptide library of claim 62, wherein said microtiter plate comprises a plurality of first and second wells, wherein:
(a) said first and second wells contain a liquid and are connected by a capillary tube fluid circuit which is also filled with a liquid;
(b) said capillary tube fluid circuit comprises a detection section in which passage of a reaction product may be detected;
(c) said first and second wells are in contact with first and second electrodes for applying an electric current across the capillary tube fluid circuit; and
(d) said electrodes are connected to a power supply.

66. A method for identifying an ion-donating or ion-accepting peptide substrate of an enzyme, comprising
(a) contacting the peptide library of claim 1 with said enzyme under conditions that allow for ion transfer to or from peptides that are substrates for said enzyme; and
(b) identifying peptides in the library that have been modified by the enzyme.

67. The method of claim 66, further comprising a step wherein the library is subjected to an electric field whereby positively charged peptides move toward a cathode, negatively charged peptides move toward an anode, and neutral or uncharged peptides do not move.

68. The method of claim 66, further comprising the step of determining the amino acid sequence of any peptide in said library that was modified by said enzyme, thereby identifying an ion-donating or ion-accepting amino acid sequence motif.

69. The method of claim 66, wherein said ion is phosphoryl, said enzyme is a kinase and peptide substrates for said kinase move toward an anode after contact with said kinase.

70. The method of claim 69, wherein said kinase is selected from the group consisting of serine, threonine, tyrosine, histidine, aspartate and lysine kinases.

71. The method of claim 69, wherein said peptide molecules in said library each have a net charge of +1 or a neutral charge.

72. The peptide library of claim 71, wherein said peptide molecules each have a net charge of +1.

73. The method of claim 67, wherein the ion is phosphoryl, said enzyme is a phosphatase and peptide substrates for said phosphatase move toward a cathode after contact with said phosphatase.

74. The method of claim 73, wherein said phosphatase is selected from the group consisting of serine, threonine, tyrosine, histidine, aspartate and lysine phosphatases.

75. The method of claim 73, wherein said peptide molecules in said library each have a net charge of −1 or a neutral charge.

76. The peptide library of claim 75, wherein said peptide molecules each have a net charge of −1.

77. The method of claim 66, wherein individual peptide molecules in said library are segregated into individual wells of one or more microtiter plates, and the amino acid sequence of peptide substrates for said enzyme are identified based on their position in the microtiter plate.

78. The method of claim 66, wherein mixtures of peptide molecules having at least one variable position are segregated into individual wells of one or more microtiter plates, and the amino acid sequence of peptide substrates for said enzyme are identified using mass spectrometry.

79. The method of claim 66, wherein peptides of said peptide library are associated with a detectable label.

80. The method of claim 79, wherein said label is a fluorophore.

81. The method of claim 79, wherein peptide substrates for said enzyme are detected following application of the electric field by means of said detectable label.

82. The method of claim 77, wherein peptide substrates for said enzyme are captured onto a matrix via electrophoretic separation.

83. The method of claim 82, wherein said peptide substrates are associated with a fluorescent label and are detected using a fluorescence plate reader.

84. The method of claim 82, wherein said microtiter plate comprises:
(a) a plurality of substantially tubular sample wells arrayed in the sample plate; and
(b) at least one capture matrix, wherein the capture matrix is disposed in each of the sample wells proximate an end of the sample wells, and wherein the capture matrix comprises a diffusion-inhibiting material; and
wherein at least one sample well is placed in electrical contact with at least one first electrode at the bottom end of the sample well, and with at least one second electrode at the top end of the sample well, wherein both electrodes are coupled to a power source.

85. The method of claim 82, wherein said microtiter plate comprises:
at least one microstructure, each microstructure comprising a series of microstructure sections and channels, wherein each microstructure section is directly interconnected to at least one other microstructure section by at least one channel, the series comprising:
at least one sample accepting microstructure section, wherein the sample accepting section is fluidly connected to the exterior of the microstructure plate;
at least one first electrode microstructure section;
at least one second electrode microstructure section;
at least one capture microstructure section containing a capture matrix,
wherein the capture microstructure section is between the first and second electrode microstructure sections in the series; and
wherein the microstructures in the microstructure plate are formed by at least two layers of material, wherein at least one layer is a sealing plate layer which seals at least one channel or microstructure section in the assembled microstructure plate.

86. The method of claim 82, wherein said microtiter plate comprises a plurality of first and second wells, wherein:
(a) said first and second wells contain a liquid and are connected by a capillary tube fluid circuit which is also filled with a liquid;
(b) said capillary tube fluid circuit comprises a detection section in which passage of a reaction product may be detected;
(c) said first and second wells are in contact with first and second electrodes for applying an electric current across the capillary tube fluid circuit; and
(d) said electrodes are connected to a power supply.

87. A method of making a peptide library of claim 1, comprising:
(a) determining the identity of peptides of a given length having the same net charge or a neutral charge; and
(b) synthesizing the peptides identified.

88. The method of claim 87, wherein the identity of peptides of a given length having the same net charge or a neutral charge is determined using an algorithm.

89. The method of claim 87, wherein said peptide molecules each have a net charge of +1.

90. The method of claim 87, wherein said peptide molecules each have a net charge of −1.

91. The method of claim 87, wherein said peptide molecules each have a net neutral charge.

92. The method of claim 87, wherein said library comprises peptide molecules having at least one variable position in relation to each other.

93. The method of claim 92, wherein said at least one variable position is filled with an amino acid selected from a subset of amino acids.

94. The method of claim 93, wherein said subset of amino acids contains one or more amino acids selected from the group consisting of basic or positively charged amino acids, acidic or negatively charged amino acids, hydrophobic amino acids, spacing or neutral amino acids, phosphoryl-accepting amino acids, phosphoryl-donating amino acids, phosphorylated amino acids and bending amino acids.

95. The method of claim 92, wherein said at least one variable position is filled with an amino acid selected from a subset of amino acids such that the number of positively charged amino acids in each peptide minus the number of negatively charged amino acids in the same peptide equals +1.

96. The method of claim 92, wherein said at least one variable position is filled with an amino acid selected from a subset of amino acids such that the number of positively charged amino acids in each peptide minus the number of negatively charged amino acids in the same peptide equals −1.

97. The method of claim 88, wherein said algorithm employs at least the following constraint: the number of positively charged amino acids taken with the number of negatively charged amino acids results in a net charge of +1.

98. The method of claim 88, wherein said algorithm employs at least the following constraint: the number of positively charged amino acids taken with the number of negatively charged amino acids results in a net charge of −1.

99. The method of claim 97, wherein the positively charged amino acids are selected from the group consisting of arginine and lysine.

100. The method of claim 99, wherein the positively charged amino acid is arginine.

101. The method of claim 97, wherein the negatively charged amino acids are selected from the group consisting of aspartic acid or glutamic acid.

102. The method of claim 101, wherein the negatively charged amino acid is glutamic acid.

103. The method of claim 98, wherein the positively charged amino acids are selected from the group consisting of arginine and lysine.

104. The method of claim 103, wherein the positively charged amino acid is arginine.

105. The method of claim 98, wherein the negatively charged amino acids are selected from the group consisting of aspartic acid or glutamic acid.

106. The method of claim 105, wherein the negatively charged amino acid is glutamic acid.

107. The method of claim 97, wherein the algorithm employs one or more of the following constraints: the number of hydrophobic amino acids is less than three and the number of prolines is less than two.

108. The method of claim 98, wherein the algorithm employs one or more of the following constraints: the number of hydrophobic amino acids is less than three and the number of prolines is less than two.

109. The peptide library of claim 13, wherein said subset of amino acids for filling variable positions comprises arginine, glutamic acid, alanine, leucine and proline.

110. The peptide library of claim 109 further comprising a non-variable position occupied by a phosphoryl-accepting amino acid or a phosphoryl-donating amino acid.

111. The peptide library of claim 110, wherein said phosphoryl-accepting or phosphoryl-donating amino acid is selected from the group consisting of serine, threonine, tyrosine, histidine, aspartate and lysine.

112. A kit for identifying a peptide substrate comprising the peptide library of claim 1.

113. The kit of claim 112, wherein individual peptide molecules or variable mixtures thereof are segregated into individual wells of one or more microtiter plates.

114. The kit of claim 113, wherein said microtiter plate comprises:
(a) a plurality of substantially tubular sample wells arrayed in the sample plate; and
(b) at least one capture matrix, wherein the capture matrix is disposed in each of the sample wells proximate an end of the sample wells, and wherein the capture matrix comprises a diffusion-inhibiting material; and
wherein at least one sample well may be placed in electrical contact with at least one first electrode at the bottom end of the sample well, and with at least one second electrode at the top end of the sample well, wherein both electrodes are coupled to a power source.

115. The kit of claim 113, wherein said microtiter plate comprises:
at least one microstructure, each microstructure comprising a series of microstructure sections and channels, wherein each microstructure section is directly interconnected to at least one other microstructure section by at least one channel, the series comprising:
at least one sample accepting microstructure section, wherein the sample accepting section is fluidly connected to the exterior of the microstructure plate;
at least one first electrode microstructure section;
at least one second electrode microstructure section;
at least one capture microstructure section containing a capture matrix,
wherein the capture microstructure section is between the first and second electrode microstructure sections in the series; and
wherein the microstructures in the microstructure plate are formed by at least two layers of material, wherein at least one layer is a sealing plate layer which seals at least one channel or microstructure section in the assembled microstructure plate.

116. The kit of claim 113, wherein said microtiter plate comprises a plurality of first and second wells, wherein:
(a) said first and second wells contain a liquid and are connected by a capillary tube fluid circuit which is also filled with a liquid;
(b) said capillary tube fluid circuit comprises a detection section in which passage of a reaction product may be detected;
(c) said first and second wells are in contact with first and second electrodes for applying an electric current across the capillary tube fluid circuit; and
(d) said electrodes are connected to a power supply.

117. The peptide library of claim 14, wherein said subset of amino acids comprises at least one phosphoryl-donating amino acid.

118. The peptide library of claim 117, wherein said phosphoryl-donating amino acid is selected from the group consisting of serine, threonine, tyrosine, histidine, aspartate and lysine.

119. The peptide library of claim 14, wherein said subset of amino acids comprises at least one phosphorylated amino acid.

120. The peptide library of claim 119, wherein said phosphorylated amino acid is selected from the group consisting of serine, threonine, tyrosine, histidine, aspartate and lysine.

121. A peptide kinase effector library comprising a plurality of different kinase effector peptides having the same motif, wherein said different peptides are segregated into reaction wells or vessels, and wherein each well or vessel further comprises a kinase substrate peptide that is associated with a detectable label, that has a +1 charge and contains a kinase phosphorylation site.

122. The peptide library of claim 121, wherein groups of effector molecules are contained in individual wells.

123. The peptide library of claim 121, wherein said effector peptides and said peptide substrate are contained on a single peptide molecule.

124. The peptide library of claim 121 wherein said kinase is selected from the group consisting of serine, threonine, tyrosine, histidine, aspartate and lysine kinases.

125. The peptide library of claim 121, wherein said detectable label is a fluorophore.

126. The peptide library of claim 125, wherein the fluorophore is selected from the group consisting of Bodipy, Texas Red, DAPI, Cy-Dyes, Lissamine, fluorescein, rhodamine, phycoerythrin, free or chelated lanthanide series salts and coumarin.

127. The peptide library of claim 121, wherein the detectable label is separated from amino acids of said peptide molecules by a linker.

128. The peptide library of claim 121, wherein the kinase effector peptide molecules comprise a linker.

129. The peptide library of claim 127, wherein said linker is selected from the group consisting of polyethylene glycol (PEG) and polysaccharides, and has a molecular weight of about 80 to 4000 Daltons.

130. The peptide library of claim 128, wherein said linker is selected from the group consisting of polyethylene glycol (PEG) and polysaccharides, and has a molecular weight of about 80 to 4000 Daltons.

131. The peptide library of claim 121, wherein the individual peptide molecules or groups thereof are segregated into individual wells of one or more microtiter plates.

132. The peptide library of claim 131, wherein said microtiter plates fit into an electrophoretic apparatus.

133. The peptide library of claim 132, wherein said microtiter plate comprises:
(a) a plurality of substantially tubular sample wells arrayed in the sample plate; and
(b) at least one capture matrix, wherein the capture matrix is disposed in each of the sample wells proximate an end of the sample wells, and wherein the capture matrix comprises a diffusion-inhibiting material; and
wherein at least one sample well may be placed in electrical contact with at least one first electrode at the bottom end of the sample well, and with at least one second electrode at the top end of the sample well, wherein both electrodes are coupled to a power source.

134. The peptide library of claim 132, wherein said microtiter plate comprises:
at least one microstructure, each microstructure comprising a series of microstructure sections and channels, wherein each microstructure section is directly interconnected to at least one other microstructure section by at least one channel, the series comprising:
at least one sample accepting microstructure section, wherein the sample accepting section is fluidly connected to the exterior of the microstructure plate;
at least one first electrode microstructure section;
at least one second electrode microstructure section;
at least one capture microstructure section containing a capture matrix,
wherein the capture microstructure section is between the first and second electrode microstructure sections in the series; and
wherein the microstructures in the microstructure. plate are formed by at least two layers of material, wherein at least one layer is a sealing plate layer which seals at least one channel or microstructure section in the assembled microstructure plate.

135. The peptide library of claim 132, wherein said microtiter plate comprises a plurality of first and second wells, wherein:
(a) said first and second wells contain a liquid and are connected by a capillary tube fluid circuit which is also filled with a liquid;
(b) said capillary tube fluid circuit comprises a detection section in which passage of a reaction product may be detected;
(c) said first and second wells are in contact with first and second electrodes for applying an electric current across the capillary tube fluid circuit; and
(d) said electrodes are connected to a power supply.

136. A method for identifying a kinase effector peptide sequence or sequence motif, comprising
(a) contacting the peptide library of claim 121 with said kinase under conditions that allow for phosphorylation of said substrate peptide where an effector for said kinase is present; and
(b) identifying substrate peptides in the library that have been phosphorylated.

137. The method of claim 136, further comprising a step wherein the library is subjected to an electric field whereby positively charged peptides move toward a cathode, negatively charged peptides move toward an anode, and neutral or uncharged peptides do not move.

138. The method of claim 136, further comprising the step of determining the amino acid sequence of any effector peptide or motif in said library that facilitated phosphorylation of said substrate peptide, thereby identifying a peptide effector for said kinase.

139. The method of claim 136, wherein said kinase is selected from the group consisting of serine, threonine, tyrosine, histidine, aspartate and lysine kinases.

140. A peptide phosphatase effector library comprising a plurality of different phosphatase effector peptides having the same motif, wherein said different peptides are segregated into reaction wells or vessels, and wherein each well or vessel further comprises a phosphatase substrate peptide that is associated with a detectable label, that has a −1 charge and contains a phosphatase dephosphorylation site.

141. The peptide library of claim 140, wherein groups of effector molecules are contained in individual wells.

142. The peptide library of claim 140, wherein said phosphatase effector peptides and said substrate peptide are contained on a single peptide molecule.

143. The peptide library of claim 140 wherein said phosphatase is selected from the group consisting of serine, threonine, tyrosine, histidine, aspartate and lysine phosphatases.

144. The peptide library of claim 140, wherein said detectable label is a fluorophore.

145. The peptide library of claim 144, wherein the fluorophore is selected from the group consisting of Bodipy, Texas Red, DAPI, Cy-Dyes, Lissamine, fluorescein, rhodamine, phycoerythrin, free or chelated lanthanide series salts and coumarin.

146. The peptide library of claim 140, wherein the detectable label is separated from amino acids of said peptide molecules by a linker.

147. The peptide library of claim 140, wherein the phosphatase effector peptide molecules comprise a linker.

148. The peptide library of claim 146, wherein said linker is selected from the group consisting of polyethylene glycol (PEG) and polysaccharides, and has a molecular weight of about 80 to 4000 Daltons.

149. The peptide library of claim 147, wherein said linker is selected from the group consisting of polyethylene glycol (PEG) and polysaccharides, and has a molecular weight of about 80 to 4000 Daltons.

150. The peptide library of claim 140, wherein the effector peptides or groups thereof are segregated into individual wells of one or more microtiter plates.

151. The peptide library of claim 150, wherein said microtiter plates fit into an electrophoretic apparatus.

152. The peptide library of claim 151, wherein said microtiter plate comprises:
(a) a plurality of substantially tubular sample wells arrayed in the sample plate; and
(b) at least one capture matrix, wherein the capture matrix is disposed in each of the sample wells proximate an end of the sample wells, and wherein the capture matrix comprises a diffusion-inhibiting material; and
wherein at least one sample well may be placed in electrical contact with at least one first electrode at the bottom end of the sample well, and with at least one second electrode at the top end of the sample well, wherein both electrodes are coupled to a power source.

153. The peptide library of claim 151, wherein said microtiter plate comprises:
at least one microstructure, each microstructure comprising a series of microstructure sections and channels, wherein each microstructure section is directly interconnected to at least one other microstructure section by at least one channel, the series comprising:
at least one sample accepting microstructure section, wherein the sample accepting section is fluidly connected to the exterior of the microstructure plate;
at least one first electrode microstructure section;
at least one second electrode microstructure section;
at least one capture microstructure section containing a capture matrix,
wherein the capture microstructure section is between the first and second electrode microstructure sections in the series; and
wherein the microstructures in the microstructure plate are formed by at least two layers of material, wherein at least one layer is a sealing plate layer which seals at least one channel or microstructure section in the assembled microstructure plate.

154. The peptide library of claim 151, wherein said microtiter plate comprises a plurality of first and second wells, wherein:
(a) said first and second wells contain a liquid and are connected by a capillary tube fluid circuit which is also filled with a liquid;
(b) said capillary tube fluid circuit comprises a detection section in which passage of a reaction product may be detected;
(c) said first and second wells are in contact with first and second electrodes for applying an electric current across the capillary tube fluid circuit; and
(d) said electrodes are connected to a power supply.

155. A method for identifying a phosphatase effector peptide sequence or sequence motif, comprising
(a) contacting the peptide library of claim 140 with said phosphatase under conditions that allow for dephosphorylation of said substrate peptide where an effector for said phosphatase is present; and
(b) identifying substrate peptides in the library that have been dephosphorylated.

156. The method of claim 155, further comprising a step wherein the library is subjected to an electric field whereby positively charged peptides move toward a cathode, negatively charged peptides move toward an anode, and neutral or uncharged peptides do not move.

157. The method of claim 155, further comprising the step of determining the amino acid sequence of any effector peptide or motif in said library that facilitated dephosphorylation of said substrate peptide, thereby identifying a peptide effector for said phosphatase.

158. The method of claim 155, wherein said phosphatase is selected from the group consisting of serine, threonine, tyrosine, histidine, aspartate and lysine phosphatases.

159. The method of claim 88, wherein the algorithm executes the following steps:
```
#include <stdio.h>
int a[6];
void print_results(void){
      int j;
      printf(“J”);
      for(j=0;j<3;j++){
            switch (a[j]){
                  case 0:
                        printf(“R”);
                        break;
                  case 1:
                        printf(“E”);
                        break;
                  case 2:
                        printf(“P”);
                        break;
                  case 3:
                        printf(“L”);
                        break;
                  case 4:
                        printf(“A”);
                        break;
            }
      }
      printf(“S”);
      for(j=3;j<6;j++){
            switch (a[j]){
                  case 0:
                        printf(“R”);
                        break;
                  case 1:
                        printf(“E”);
                        break;
                  case 2:
                        printf(“P”);
                        break;
                  case 3:
                        printf(“L”);
                        break;
                  case 4:
                        printf(“A”);
                        break;
            }
      }
      printf(“G♯n”);
}
void main (void){
      int i = 0, ii=0, iii=0, iiii=0, iiiii=0;
      int i1,i2,i3,i4,i5,i6;
      int nL = 0;
      int nP = 0;
      int nC = 0;
      int nR = 0;
      int j;
      for(i1=0; i1<5; i1++){
            a[0] = i1;
// if(2 == i1){nP = 1;}else{nP = 0;}
            for (i2=0; i2<5; i2++){
                  a[1] = i2;
                  for (i3=0; i3<5; i3++){
                        a[2] = i3;
                        for (i4=0; i4<5; i4++){
                              a[3] = i4;
                              for (i5=0; i5<5; i5++){
                                    a[4] = i5;
                                    for (i6=0; i6<5; i6++){
                                          a[5] = i6;
                                          i++;
                                          nP = 0;
                                          for(j = 0; j<6; j++){
                                                if(a[j] == 2){nP++;}
                                          }
                                          if(nP < 2){ //nP
                                                ii++;
                                                nL = 0;
                                                for(j = 0; j<6; j++){
                                                      if(a[j] == 3){nL++;}
                                                }
                                                if(nL < 3){
                                                      iii++;
                                                      nC = 0;
                                                      for(j = 0; j<6; j++){
                                                            if(a[j] ==
0){nC++;}
                                                            if(a[j] == 1){nC−
−;}
                                                      }
                                                      if(nC == 1){
                                                            iiii++;
                                                            nR = 0;
                                                            for(j = 0; j<6;
j++){
                                                                  if(a[j] ==
0){nR++;}
                                                            }
                                                            if(nR < 3){
      print_results( );
                                                                  iiiii++;
                                                            }
                                                      }
                                                }
                                          }
                                    }
                              }
                        }
                  }
            }
      }
// printf(“i = %d♯n”, i);
// printf(“ii = %d♯n”, ii);
// printf(“iii = %d♯n”, iii);
// printf(“iiii = %d♯n”, iiii);
// printf(“iiiii = %d♯n”, iiiii);
}.
```

160. A computer readable medium storing computer executable instructions for designing the peptide library of claim 1.

161. The computer readable medium of claim 160, wherein the identity of peptides in the library is determined using an algorithm.

162. The computer readable medium of claim 161, wherein said algorithm executes the following steps:
```
#include <stdio.h>
int a[6];
void print_results (void){
      int j;
      printf(“J”);
      for(j=0;j<3;j++){
            switch (a[j]){
                  case 0:
                        printf(“R”);
                        break;
                  case 1:
                        printf(“E”);
                        break;
                  case 2:
                        printf(“P”);
                        break;
                  case 3:
                        printf(“L”);
                        break;
                  case 4:
                        printf(“A”);
                        break;
            }
      }
      printf(“S”);
      for(j=3;j<6;j++){
            switch (a[j]) {
                  case 0:
                        printf(“R”);
                        break;
                  case 1:
                        printf(“E”);
                        break;
                  case 2:
                        printf(“P”);
                        break;
                  case 3:
                        printf(“L”);
                        break;
                  case 4:
                        printf(“A”);
                        break;
            }
      }
      printf(“G♯n”);
}
void main(void){
      int i = 0, ii=0, iii=0, iiii=0, iiiii=0;
      int i1,i2,i3,i4,i5,i6;
      int nL = 0;
      int nP = 0;
      int nC = 0;
      int nR = 0;
      int j;
      for (i1=0; i1<5; i1++){
            a[0] = i1;
// if(2 == i1){nP = 1;}else{nP = 0;}
            for (i2=0; i2<5; i2++){
                  a[1] = i2;
                  for (i3=0; i3<5; i3++){
                        a[2] = i3;
                        for (i4=0; i4<5; i4++){
                              a[3] = i4;
                              for (i5=0; i5<5; i5++){
                                    a[4] = i5;
                                    for (i6=0; i6<5; i6++){
                                          a[5] = i6;
                                          i++;
                                          nP = 0;
                                          for(j = 0; j<6; j++){
                                                if(a[j] == 2){nP++;}
                                          }
                                          if(nP < 2){ //nP
                                                ii++;
                                                nL = 0;
                                                for(j = 0; j<6; j++){
                                                      if(a[j] == 3){nL++;}
                                                }
                                                if(nL < 3){
                                                      iii++;
                                                      nC = 0;
                                                      for(j = 0; j<6; j++){
                                                            if(a[j] ==
0){nC++;}
                                                            if(a[j] == 1){nC−
−;}
                                                      }
                                                      if(nC == 1){
                                                            iiii++;
                                                            nR = 0;
                                                            for(j = 0; j<6;
j++){
                                                                  if(a[j] ==
0){nR++;}
                                                            }
                                                            if(nR < 3){
      print_results( );
                                                                  iiiii++;
                                                            }
                                                      }
                                                }
                                          }
                                    }
                              }
                        }
                  }
            }
      }
// printf(“i = %d♯n”, i);
// printf(“ii = %d♯n”, ii);
// printf(“iii = %d♯n”, iii);
// printf(“iiii = %d♯n”, iiii);
// printf(“iiiii = %d♯n”, iiiii);
}.
```
